(19) United States
(12) Patent Application Publication
(10) Pub. No.: US 2026/0194525 A1
(43) Pub. Date: Jul. 9, 2026

(54) METHOD FOR DETECTING CEACAM1

(71) Applicant: Advanced Life Science Institute, Inc., Tokyo (JP)

(72) Inventors: Osamu ARAI, Tokyo (JP); Shintaro YAGI, Tokyo (JP); Katsumi AOYAGI, Tokyo (JP)

(73) Assignee: Advanced Life Science Institute, Inc., Tokyo (JP)

(21) Appl. No.: 19/101,477

(22) PCT Filed: Aug. 7, 2023
(86) PCT No.: PCT/JP2023/028802

§ 371 Date: Feb. 5, 2025

(30) Foreign Application Priority Data
Aug. 8, 2022 (JP) 2022-126644

(51) Int. Cl. G01N 33/57525; G01N 33/57535; G01N 33/68

(52) U.S. Cl. CPC G01N 33/57525; G01N 33/57535; G01N 33/6878; G01N 2333/70503; G01N 2458/00; G01N 2470/04

(57) ABSTRACT

A technique for detecting a specific protein that is suitable as a cancer marker with high sensitivity. This technique includes a method for detecting CEACAM1, which involves denaturing CEACAM1 in a CEACAM1-containing specimen to produce a denatured CEACAM1; and detecting CEACAM1 by a sandwich assay using both a capture antibody and a labeled antibody that have an ability to recognize the denatured CEACAM1.

### FIELD

The present invention relates to a method for detecting CEACAM1, and the like.

### BACKGROUND

CEACAM1 is a protein belonging to the carcinoembryonic antigen (CEA) family. CEACAM1 has 12 isoforms, of which 3 are known to be secreted and present in body fluids such as serum. In addition, it is known that secreted CEACAM1 can be used as a marker for cancer such as melanoma, pancreatic cancer, and urothelial bladder cancer. In pancreatic cancer, CEACAM1 has been confirmed to exhibit higher diagnostic sensitivity and diagnostic specificity than CA19-9, a general-purpose cancer marker. Therefore, CEACAM1 is expected as a cancer marker.

Several prior arts have been published for CEACAM1. For example, Patent Literature 1 discloses an antibody (optionally an antibody capable of binding to other subtypes of the CEACAM protein family) having the ability to recognize an epitope specific to CEACAM1. Patent Literature 2 discloses a method for diagnosing the presence or absence of extrahepatic bile duct cancer, intrahepatic bile duct cancer, or gallbladder cancer using an antibody having the ability to specifically bind to CEACAM1. Patent Literature 3 discloses a method in which human serum or plasma under acidic conditions is heated to separate unnecessary substances, and then carcinoembryonic antigen (CEA) is detected by immunoassay.

### CITATION LIST

### Patent Literature

Patent Literature 1: JP 2015-502138 A
Patent Literature 2: JP 2018-17723 A
Patent Literature 3: JP H02-503716 A

### SUMMARY

### Technical Problem

An object of the present invention is to detect a specific protein that can be used as a cancer marker with high sensitivity.

Another object of the present invention is to detect a specific protein that can be used as a cancer marker with high sensitivity and specificity.

As a result of intensive studies, the present inventors have found that CEACAM1 that can be used as a cancer marker can be detected with high sensitivity (and specificity) by using a sandwich assay using both a capture antibody and a labeled antibody that have the ability to recognize a denatured CEACAM1 after denaturation of CEACAM1 while selecting CEACAM1 as a specific protein that can be used as a cancer marker, and have completed the present invention. The prior art neither describes nor suggests using such a sandwich assay after denaturation of CEACAM1.

That is, the present invention is as follows.
[1] A method for detecting CEACAM1, comprising:
   (1) denaturing CEACAM1 in a CEACAM1-containing specimen to produce a denatured CEACAM1; and
   (2) detecting CEACAM1 by a sandwich assay using both a capture antibody and a labeled antibody that have an ability to recognize the denatured CEACAM1.
[2] The method according to [1], wherein the denaturing is selected from the group consisting of a denaturant, heating, and a combination thereof.
[3] The method according to [2], wherein the denaturant is selected from the group consisting of an alkaline substance, a surfactant, and a reducing agent, and a combination of two or more thereof.
[4] The method according to any one of [1]-[3], wherein an antibody having an ability to recognize an epitope represented by the amino acid sequence of PANSGRETIY (SEQ ID NO: 1) in CEACAM1 is used as one of the capture antibody and the labeled antibody.
[5] The method according to [4], wherein an antibody having an ability to recognize an epitope represented by the amino acid sequence of TESMP (SEQ ID NO: 2) in CEACAM1 is further used as one of the capture antibody and the labeled antibody.
[6] The method according to [4] or [5], wherein an antibody having an ability to recognize an epitope represented by the amino acid sequence of DTTYLWWINN (SEQ ID NO: 3) in CEACAM1 is used as the other of the capture antibody and the labeled antibody.
[7] The method according to [4] or [5], wherein an antibody having an ability to recognize an epitope represented by the amino acid sequence of EATGQFHVYP (SEQ ID NO: 4) in CEACAM1 is used as the other of the capture antibody and the labeled antibody.
[8] A method of detecting a cancer, comprising:
   (1) measuring a CEACAM1 amount in a specimen collected from a subject by the method according to any of [1] to [7]; and
   (2) comparing the measured CEACAM1 amount to a reference value.
[9] The method according to [8], wherein the cancer is selected from the group consisting of colon cancer, liver cancer, and pancreatic cancer, and a combination of two or more thereof.
[10] A method for treating a cancer, comprising the steps of:
   (1) measuring a CEACAM1 amount in a specimen collected from a subject by the method according to any of [1] to [7];
   (2) comparing the measured CEACAM1 amount to a reference value;
   (3) selecting a subject in which a CEACAM1 amount higher than a reference value has been measured; and
   (4) administering an anti-cancer agent to the selected subject.
[11] A kit for CEACAM1 detection, comprising:
   (1) a denaturant;
   (2) a capture antibody having an ability to recognize a denatured CEACAM1; and
   (3) a labeled antibody having an ability to recognize a denatured CEACAM1.

### Effects of Invention

The method for detecting CEACAM1 of the present invention is useful, for example, for detection of CEACAM1 with high sensitivity and specificity.

The method for detecting cancer of the present invention is useful for detecting cancer with high diagnostic sensitivity and high diagnostic specificity because CEACAM1 that is a cancer marker can be measured with high sensitivity and specificity.

The treatment therapy of the present invention is useful for effective treatment with an anti-cancer agent.

In addition, according to these methods of the present invention, the influence of autoantibodies can be reduced or avoided.

The kit of the present invention is useful, for example, for convenient implementation of the method of the present invention.

### BRIEF DESCRIPTION OF DRAWINGS

FIG. 1 is a view illustrating an amino acid sequence of an expressed protein (pAT-TrpE-His-CEACAM1-N, pAT-TrpE-His-CEACAM1-N-m3C3d, pAT-TrpE-His-CEACAM1-A1, and pAT-TrpE-His-CEACAM1-A1-m3C3d).

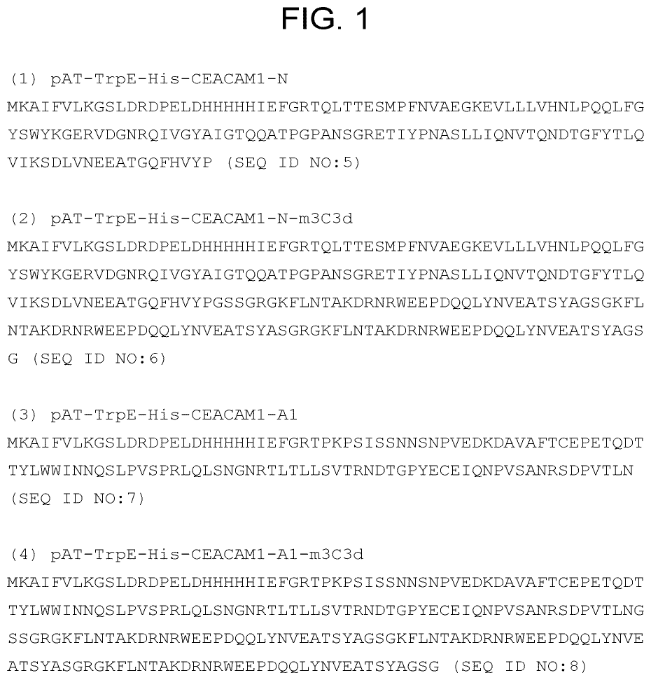

FIG. 2 is a view illustrating cross-reactivity of an anti-CEACAM1 monoclonal antibody.

FIG. 3 is a view illustrating an amino acid sequence alignment of CEACAM family. Amino acid sequence alignment from N domain to A1 domain including CEACAM family signal sequence searched by UniProt database. Amino acids with high homology are shown in black outline. The epitope of each antibody was shown on the sequence of CEACAM1, and the amino acids considered as hot spots of each antibody epitope were shown in black frames.

FIG. 4 is a view illustrating blood concentration distributions of various cancer specimens. Colon cancer specimens (Colon cancer, n=42), liver cancer specimens (Liver cancer, n=50), pancreatic cancer specimens (Pancreatic cancer: n=40), and healthy human specimens (Normal, n=36).

FIG. 5A is a view illustrating receiver operating characteristic (ROC) analysis in a colon cancer specimen.

FIG. 5B is a view illustrating ROC analysis in liver cancer specimens.

FIG. 5C is a view illustrating ROC analysis in a pancreatic cancer specimen.

FIG. 6 is a view illustrating a difference in effect depending on the presence or absence of SDS reduction heat treatment. CEACAM1 antigen (1 μg/mL) without denaturation treatment (SDS reduction heat treatment) or under SDS reduction heat treatment conditions was reacted with a co-immobilized plate of A3054 and A3073, and the result of detection with an ALP-labeled A7004 antibody is shown.

FIG. 7 is a view illustrating an effect (part 1) of specimen denaturation treatment by alkali treatment (NaOH).

FIG. 8 is a view illustrating the effect (part 2) of the test denaturation treatment by the alkali treatment (NaOH).

FIG. 9 is a view illustrating blood concentration distributions of various cancer specimens. Colon cancer specimens (Colon cancer, n=42), liver cancer specimens (Liver cancer, n=50), pancreatic cancer specimens (Pancreatic cancer: n=40), and healthy human specimens (Normal, n=36).

FIG. 10A is a view illustrating ROC analysis of a colon cancer specimen for a healthy human specimen.

FIG. 10B is a view illustrating ROC analysis of liver cancer specimens in healthy humans.

FIG. 10C is a view illustrating ROC analysis of pancreatic cancer specimens in healthy humans.

FIG. 11 is a view illustrating a correlation between SDS reduction heat treatment and alkali treatment.

FIG. 12 is a view illustrating an effect of denaturation treatment on an autoantibody model specimen. A) A measurement system including no denaturation treatment, and B) a measurement system including a denaturation treatment.

### EMBODIMENTS FOR CARRYING OUT THE INVENTION

The present invention relates to a method for detecting CEACAM1, including the steps of:
(1) denaturing CEACAM1 in a CEACAM1-containing specimen to produce a denatured CEACAM1; and
(2) detecting CEACAM1 by a sandwich assay using both a capture antibody and a labeled antibody that have an ability to recognize the denatured CEACAM1.

In the step (1), CEACAM1 is denatured in the CEACAM1-containing specimen, thereby producing a denatured CEACAM1.

In the present invention, denaturation is carried out such that linear epitopes of CEACAM1 that cannot be exposed in the native state (that is, a non-denatured state) are exposed in order to improve recognition by the antibody. For example, when CEACAM1 is in an aggregated state or in a multimer or complex form, the linear epitope is covered, so that the linear epitope cannot be sufficiently exposed.

The denaturation can be performed by a denaturant, heating, or a combination thereof. Examples of the denaturant include an alkaline substance, a surfactant, a chaotropic denaturant (for example, urea, guanidine), a reducing agent, and a combination of two or more thereof.

Examples of the alkaline substance include inorganic substances (for example, metal ions) and organic substances. As the alkaline substance, many substances exist, and among them, hydroxides of alkali metals (for example, sodium, potassium) or alkaline earth metals (for example, magnesium, calcium) are generally used. Therefore, such a hydroxide can be suitably used.

The concentration of the alkaline substance is not particularly limited as long as CEACAM1 can be denatured, and may be, for example, a concentration of 0.1 M or more, preferably 0.2 M or more, more preferably 0.3 M or more, and still more preferably 0.35 M or more. The concentration of the alkaline substance may also be 5 M or less, 2 M or less, or 1 M or less.

The alkaline substance may be used with a surfactant. As the surfactant, a nonionic surfactant, an anionic surfactant, a cationic surfactant, and an amphoteric surfactant are preferable. In particular, the nonionic surfactant is preferred. Examples of the nonionic surfactant include ether type surfactants such as polyoxyethylene (23) lauryl ether (Brij35) and polyethylene glycol hexadecyl ether (Brij58); polyoxyethylene sorbitan fatty acid esters (For example, Tween (trade name, registered trademark) series) such as polyoxyethylene sorbitan monolaurate (Tween20) and polyoxyethylene sorbitan monopalmitate (Tween40) polyoxyethylene sorbitan monooleate (Tween80); polyoxyethylene alkylphenyl ethers (For example, Triton (trade name, registered trademark) series) such as polyoxyethylene octyl phenyl ether; secondary alcohol polyoxyethylene ethers (For example, Tergitol (trade name, registered trademark) series) such as Tergitol (registered trademark) 15-S-7; and copolymers of ethylene oxide and propylene oxide (For example, Pluronic (trade name, registered trademark) series) such as poly(ethylene glycol)-block-poly(propylene glycol)-block-poly(ethylene glycol) (PluronicF108). The concentration of the surfactant is not particularly limited, and can be appropriately set.

In the case of denaturation with a denaturation treatment liquid containing an alkaline substance, a step of adding a neutralizing liquid containing an acidifying agent or a buffer having buffering ability in order to neutralize or adjust the pH after the denaturation treatment may be further included. Examples of the acidifying agent include hydrochloric acid, sulfuric acid, and acetic acid. When the step of adding a neutralizing liquid is not included, for example, in the reaction step described below, the alkaline substance of the denaturation treatment liquid may be neutralized by adjusting the buffering capacity, pH, and the like of the buffer to be mixed, and the influence in the reaction step may be alleviated. The pH in the reaction step can be appropriately set depending on the components contained in the reaction step, and for example, the addition amount of an acidifying agent, a buffer, or the like contained in the liquid in the reaction step can be appropriately set so as to have a pH of 5.5 to 9.5.

Examples of the surfactant include an anionic surfactant, a cationic surfactant, an amphoteric surfactant, and a nonionic surfactant. As the surfactant, one kind or two or more kinds of surfactants can be used. As the surfactant, an anionic surfactant may be used alone, or an anionic surfactant may be used in combination of an anionic surfactant and other surfactants. Examples of the anionic surfactant include a sulfuric acid ester type surfactant (for example, sodium dodecyl sulfate (SDS)), a carboxylic acid type surfactant (for example, sodium N-decanoyl sarcosine (NDS), sodium N-lauroyl sarcosine hydrate (NLS)), a sulfonic acid type surfactant (for example, sodium 1-nonanesulfonate (NSS), sodium dodecylbenzenesulfonate (SDBS)), and a carboxylic acid-sulfonic acid type surfactant (for example, sodium chondroitin sulfate (CSSS)). Examples of the amphoteric surfactant include C10APS (N-decyl-N, N-dimethyl-3-ammonio-1-propanesulfonate), C12APS (N-dodecyl-N, N-dimethyl-3-ammonio-1-propanesulfonate), C14APS (N-tetradecyl-N, N-dimethyl-3-ammonio-1-propanesulfonate), and C16APS (N-hexadecyl-N, N-dimethyl-3-ammonio-1-propanesulfonate). Examples of the cationic surfactant include decyltrimethylammonium chloride, dodecyltrimethylammonium chloride, tetradecyltrimethylammonium chloride, hexadecyltrimethylammonium chloride (C16TAC), decyltrimethylammonium bromide, dodecyltrimethylammonium bromide, tetradecyltrimethylammonium bromide, hexadecyltrimethylammonium bromide (CTAB), laurylpyridinium chloride, tetradecylpyridinium chloride, and cetylpyridinium chloride.

The concentration of the surfactant is not particularly limited as long as CEACAM1 can be denatured, and may be, for example, a concentration of 0.5 wt % or more, preferably 1 wt % or more, more preferably 2 wt % or more, and still more preferably 5 wt % or more. The concentration of surfactant may also be at a concentration of 20 wt % or less, or 15 wt % or less.

As the reducing agent, a reagent capable of cleaving a disulfide bond present in CEACAM1 can be used. Examples of such a reducing agent include tricarboxylethylphosphine (TCEP), cysteine, dithiothreitol, reduced glutathione, and β-mercaptoethanol. Examples of the protease include trypsin, chymotrypsin, Lys-C, Asp-N, Glu-C, Arg-C, asparaginyl endopeptidase, arginyl endopeptidase, and V8 protease.

The concentration of the reducing agent is not particularly limited as long as the disulfide bond present in CEACAM1 can be cleaved, and may be, for example, a concentration of 0.5 mM or more, preferably 1 mM or more, more preferably 2 mM or more, and still more preferably 5 mM or more. The concentration of the reducing agent may also be a concentration of 100 mM or less, or 50 mM or less.

The value of the concentration of the denaturant (alkaline substances, surfactants and reducing agents) is a value based on the weight or volume of the denaturation treatment liquid containing the denaturant. In addition to the denaturant, the denaturation treatment liquid contains a solvent or a dispersion medium (for example, water) and, if necessary, an additive. Examples of the additive include chelating agents such as EDTA.

The heating may be, for example, 35° C. or more, 40° C. or more, 45° C. or more, 50° C. or more, 55° C. or more, 60° C. or more, 65° C. or more, 70° C. or more, 75° C. or more, 80° C. or more, 85° C. or more, 90° C. or more, 95° C. or more, or about 100° C. (That is, boiling). The heating may also be at or below 100° C.

In certain embodiments, the denaturation may be carried out in combination with a surfactant and a reducing agent. The denaturation may also be carried out by combining a surfactant, a reducing agent and heating (for example, SDS reduction heat treatment).

The treatment time (for example, incubation time and heat treatment time after addition of denaturant to specimen) for denaturing CEACAM1 varies depending on factors such as the type and degree of denaturation treatment (for example, concentration, heating temperature), and may be, for example, 1 second or more, 5 seconds or more, 10 seconds or more, preferably 30 seconds or more, more preferably 1 minute or more, still more preferably 2 minutes or more, 3 minutes or more, or 5 minutes or more. Such time may also be 1 hour or less, 30 minutes or less, or 20 minutes or less.

The CEACAM1-containing specimen is any specimen containing CEACAM1. Preferably, the CEACAM1-containing specimen is a biological liquid specimen. The CEACAM1-containing specimen may be subjected to other treatment(s) before being used in the method of the present invention. Examples of such treatment include centrifugation, extraction, filtration, precipitation, heating, freezing, refrigeration, and stirring.

In one embodiment, the CEACAM1-containing specimen may be an animal-derived liquid specimen. Examples of the animal from which the liquid specimen is derived include animals such as mammals (for example, humans, primates such as monkeys; rodents such as mice, rats, and rabbits; ungulates such as cows, pigs, goats, horses, and sheep; and meats such as dogs and cats; aves (for example, chickens)). Preferably, the animal is a mammal, preferably a human. The animal-derived liquid specimen may be a body fluid specimen derived from an animal as described above. Examples of the body fluid specimen include blood specimens (for example, whole blood, serum and plasma), urine, saliva, lymph, tissue fluid, cerebrospinal fluid, ascites, sweat, semen, tears, mucus, milk, pleural fluid, bronchoalveolar lavage fluid, and amniotic fluid. Preferably, the body fluid is a blood specimen, urine, or saliva.

In another embodiment, the CEACAM1-containing specimen may be a culture supernatant specimen. The culture supernatant specimen may be a cell culture supernatant specimen or a tissue culture supernatant specimen. Examples and preferred examples of animals from which cells or tissues to be cultured are as described above.

CEACAM1 can be identified by the type of animal from which it is derived. Examples and preferred examples of animals from which CEACAM1 is derived are similar to the above-described animals. Thus, CEACAM1 is mammalian CEACAM1, preferably human CEACAM1. CEACAM1 also has 12 isoforms, 3 of which are secreted (CEACAM1-4C1, CEACAM1-3, and CEACAM1-3C2). Therefore, when a body fluid specimen is used, CEACAM1 may contain such a secreted CEACAM1.

After the step (1), the specimen containing the denatured CEACAM1 may be diluted with a solution (for example, a buffer). Thereby, when the specimen obtained in the step (1) contains a denaturant at a high concentration, the concentration of the denaturant can be diluted, and the sandwich assay in the step (2) can be favorably performed.

In the step (2), CEACAM1 is detected by a sandwich assay. In the sandwich assay, both a capture antibody and a labeled antibody that have the ability to recognize a denatured CEACAM1 are used. The sandwich assay can be performed in any manner using such antibodies. Such modalities include, for example, chemiluminescence immunoassay (CLIA) [for example, chemiluminescent enzyme Immunoassay (CLEIA)], enzyme immunoassay (EIA), radioimmunoassay (RIA), fluorescence immunoassay (FIA), and immunochromatography methods.

The capture antibody may be immobilized on a solid phase. The antibody against CEACAM1 may be an antibody that can be immobilized on a solid phase in the reaction step. In this specification, an antibody immobilized on a solid phase, and an antibody that can be immobilized on a solid phase in a reaction step, may be simply referred to as immobilized antibody. The capture antibody is an antibody for capturing a target antigen (In the case of the invention, denatured CEACAM1) on a solid phase. Examples of the solid phase include particles (for example, sepharose beads, agarose beads, magnetic particles), a support (for example, a membrane), and a container (for example, plates such as a plastic plate, a tube, and microchannels). For immobilization of the antibody to the solid phase, a previously known method can be used. For example, it can be performed covalently or non-covalently by a physical adsorption method, a covalent binding method, a method using an affinity substance (for example, biotin, streptavidin), and an ionic binding method. The capture antibody may be an antibody in which one type of capture antibody is immobilized on one type of solid phase (for example, particles such as magnetic particles), an antibody in which two or more types of capture antibodies are immobilized on one solid phase (for example, particles such as magnetic particles), or an antibody in which two or more types of capture antibodies are immobilized on two or more types of solid phases (for example, particles such as magnetic particles).

The labeled antibody is an antibody labeled with a labeling substance. Examples of the labeling substance include an enzyme (for example, peroxidase, alkaline phosphatase, luciferase, β-galactosidase), an affinity substance (for example, streptavidin, biotin), a fluorescent substance or a fluorescent protein (for example, fluorescein, fluorescein isothiocyanate, rhodamine, green fluorescent protein, red fluorescent protein), a luminescent or light-absorbing substance (for example, luciferin, acridinium, ruthenium), and a radioactive substance (for example, ³H, ¹⁴C, ³²P, ³⁵S, ¹²⁵I). The labeling of the antibody with the labeling substance can be performed covalently or non-covalently.

In the present invention, an antibody is a protein containing a moiety capable of binding to a target antigen (for example, variable region or corresponding portion). Therefore, examples of the antibody in the capture antibody and the labeled antibody include full-length antibodies (for example, IgG, IgM, IgA, IgD, IgE, IgY), antigen-binding fragments of antibodies (for example, F(ab′)₂, Fab′, Fab, Fv), and modified antibodies such as single-chain antibodies. The antibody may also be a polyclonal antibody or a monoclonal antibody.

In the present invention, as the capture antibody and the labeled antibody, an antibody having the ability to recognize a denatured CEACAM1 can be used. The antibody having the ability to recognize a denatured CEACAM1 may be an antibody having an ability to specifically recognize a denatured CEACAM1 or an antibody having an ability to non-specifically recognize a denatured CEACAM1. Examples of the antibody having the ability to non-specifically recognize a denatured CEACAM1 include (i) an antibody having the ability to recognize not only a denatured CEACAM1 but also native (That is, non-denaturation) CEACAM1, (ii) an antibody having the ability to recognize not only a denatured CEACAM1 but also other CEACAM1 (for example, CEACAM3), and (iii) an antibody having the ability to recognize not only a denatured CEACAM1 but also native (That is, non-denaturation) CEACAM1 and other CEACAM1 (for example, CEACAM3). At least one of the capture antibody and the labeled antibody is preferably an antibody having the ability to specifically recognize a denatured CEACAM1.

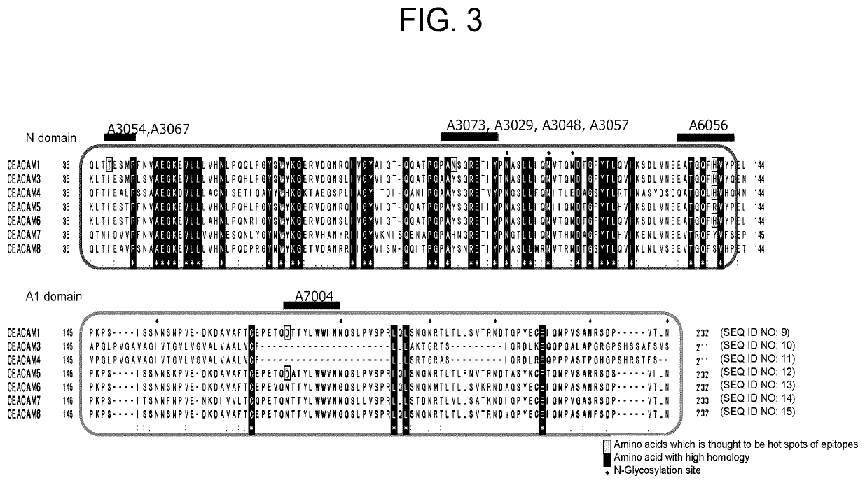

In the present invention, as the capture antibody and the labeled antibody as described above, (a) an antibody having an ability to recognize an epitope represented by the amino acid sequence of PANSGRETIY (SEQ ID NO: 1) in CEACAM1, (b) an antibody having an ability to recognize an epitope represented by the amino acid sequence of TESMP (SEQ ID NO: 2) in CEACAM1, (c) an antibody having an ability to recognize an epitope represented by the amino acid sequence of DTTYLWWINN (SEQ ID NO: 3) in CEACAM1, or (d) EATGQFHVYP (SEQ ID NO: 4) in CEACAM1 may be used. All of these epitopes are epitopes that react better to a denatured CEACAM1 than a non-denatured CEACAM1 because they are particularly exposed by denaturation as described above. According to a test using a peptide represented by the amino acid sequence of TESMP (SEQ ID NO: 2), the antibody of the above (b) above cross-reacts not only with CEACAM1 but also with CEACAM3, CEACAM5, and/or CEACAM6, and is therefore not a CEACAM1-specific antibody (see Examples and FIG. 3). However, the present inventors have found that detection sensitivity of CEACAM1 is improved by performing a sandwich assay using the antibody of the above (b) in an auxiliary manner. In addition, it has been confirmed that detection of CEACAM3, CEACAM5, and/or CEACAM6 can be avoided by using another antibody having the ability to recognize CEACAM1 in combination with the antibody of the above (b) (see Examples).

In a specific embodiment, the antibody of the above (a) or a combination of the antibody of the above (a) and the antibody of the above (b) may be used as one of the capture antibody and the labeled antibody, and the antibody of the above (c), the antibody of the above (d) or a combination of the antibody of the above (c) and the antibody of the above (d) may be used as the other. This allows CEACAM1 to be specifically and highly sensitively detected. Preferably, the antibody of the above (a) or a combination of the antibody of the above (a) and the antibody of the above (b) may be used as the capture antibody, and the antibody of the above (c), the antibody of the above (d), or a combination of the antibody of the above (c) and the antibody of the above (d) may be used as the labeled antibody.

The weight ratio of (a):(b) in the case of combining the antibody of the above (a) with the antibody of the above (b) may be, for example, 1:9 to 9:1, preferably 2:8 to 8:2, more preferably 3:7 to 7:3, still more preferably 4:6 to 6:4, and most preferably 4.5:5.5 to 5.5:4.5.

An antibody having the ability to recognize an epitope as described above can be produced by using CEACAM1 or a partial protein thereof as an antigen. In fact, the hybridomas producing the antibodies of the above (a), (b), (c) and (d) have been successfully produced with probabilities of 2/500 (0.4%), 4/500 (0.8%), 1/800 (0.1%) and 1/120 (0.8%), respectively. In addition, by using a peptide composed of an epitope recognized by the antibodies of the above (a), (b), (c), and (d) or a polypeptide rich in the peptide (polypeptide including a plurality of the epitopes) as an antigen (for example, fusion to a carrier protein such as albumin), a hybridoma producing an antibody having the ability to recognize the epitope can be efficiently produced. Alternatively, an antibody having the ability to recognize the epitope can be efficiently obtained by selecting the antibody from an antibody library using the epitope.

The invention also relates to a method for detecting a cancer, including the steps of:
(1) measuring a CEACAM1 amount in a specimen collected from a subject by the above-described method for detecting CEACAM1; and
(2) comparing the measured CEACAM1 amount to a reference value.

The step (1) in the method for detecting cancer can be performed in the same manner as the method for detecting CEACAM1 described above. The subject from which the specimen is collected is the same as the above-described animal, preferably a mammal, and more preferably a human. The specimen is the same as the animal-derived liquid specimen (for example, body fluid specimen) described above.

In the step (2) in the method for detecting cancer, the measured CEACAM1 amount is compared with a reference value. If the measured CEACAM1 amount is higher than a reference value, it can be used as an indicator that a subject may have a cancer. It is known that CEACAM1 such as secreted CEACAM1 can be used as a marker for various cancers such as melanoma, pancreatic cancer, urothelial bladder cancer, lung cancer, gastric cancer, large bowel cancer, pancreatic cancer, prostate cancer, bladder cancer, and melanoma. Therefore, the method for detecting cancer of the present invention is useful for detecting such various cancers (for example, see colon cancer, liver cancer, and pancreatic cancer shown in the Examples, as well as combinations of two or more of these).

The present invention further provides a method for treating a cancer including the steps of:
(1) measuring a CEACAM1 amount in a specimen collected from a subject by the above-described method for detecting CEACAM1;
(2) comparing the measured CEACAM1 amount to a reference value;
(3) selecting a subject in which a CEACAM1 amount higher than a reference value has been measured; and
(4) administering an anti-cancer agent to a selected subject.

The steps (1) and (2) in the method for treating a cancer can be performed in the same manner as the steps (1) and (2) in the method for detecting cancer.

The step (3) in the method for treating a cancer can be performed by selecting a subject whose CEACAM1 amount higher than the reference value has been measured based on the comparison result of the above (2).

In the step (4) in the method for treating a cancer, an anti-cancer agent is administered to a selected subject. The anti-cancer agent to be administered can be appropriately determined according to the type of cancer. In addition, other therapeutic methods such as radiation therapy may be used in combination.

The present invention further provides a kit for CEACAM1 detection, including:
(1) a denaturant;
(2) a capture antibody having an ability to recognize a denatured CEACAM1; and
(3) a labeled antibody having an ability to recognize a denatured CEACAM1.

The denaturant, the capture antibody having the ability to recognize a denatured CEACAM1, and the labeled antibody having the ability to recognize a denatured CEACAM1 are the same as those described above. The capture antibody having the ability to recognize a denatured CEACAM1 and the labeled antibody having the ability to recognize a denatured CEACAM1 may be provided in the form of a reagent for a sandwich assay using these antibodies. The kit of the present invention is useful, for example, for simple and rapid implementation of the method of the present invention.

### EXAMPLES

The present invention is described in more detail with reference to the following Examples, and the present invention is not limited to the following Examples. Note that % added to a component in Examples described later means wt %.

### Example 1: Expression and Purification of Immune Antigen and Antigen for Screening

### (A) Construction of Antigen Expression Plasmids

An antigen expression plasmid was constructed to prepare TrpE-His-CEACAM1-N-m3C3d and TrpE-His-CEACAM1-A1-m3C3d as antigens for immunization, and TrpE-His-CEACAM1-N and TrpE-His-CEACAM1-A1 as antigens for screening. Using cDNA corresponding to human CEACAM1 as a template, PCR was performed using two primers 5′-GACTCGTACGCAGCTCACTACTGAATCCATG-3′ (SEQ ID NO: 16) and 5′-GACTGTCGACTTACGGGTATACATGGAACTGTCC-3′ (SEQ ID NO: 17) to construct the pAT-TrpE-His-CEACAM1-N expression plasmid, two primers (5′-GACTCGTACGCAGCTCACTACTGAATCCATG-3′ (SEQ ID NO: 18) and 5′-GACTCTCGAGCCCGGGTATACATGGAACTGTCC-3′ (SEQ ID NO: 19) to construct the pAT-TrpE-His-CEACAM1-N-m3C3d expression plasmid, two primers GACTCGTACGCCCAAGCCCTCCATCTCCAGC-3′ (SEQ ID NO: 20) and GACTGTCGACTTAATTCAAGGTGACTGGGTCACT-3′ (SEQ ID NO: 21) to construct the expression plasmid, and two primers GACTCGTACGCCCAAGCCCTCCATCTCCAGC-3′ (SEQ ID NO: 22) and GACTCTCGAGCCATTCAAGGTGACTGGGTCACT-3′ (SEQ ID NO: 23) to construct the pAT-TrpE-His-CEACAM1-A1-m3C3d expression plasmid. PCR was performed using a KAPA Taq Extra kit (Nippon Genetics Co., Ltd.) under the following conditions: DNA denaturation at 95° C. for 15 seconds, annealing at 55° C. for 15 seconds, and DNA synthesis at 72° C. for 45 seconds, for 25 cycles. The resulting DNA fragments were separated by 1.2% agarose gel electrophoresis and purified using Freeze 'N Squeeze™ DNA Gel Extraction Spin Columns (BIO-RAD). 0.5 μg of this amplified CEACAM1 gene fragment was digested at 37° C. for 1 hour in a solution obtained by adding 10 units of BsiWI and 10 units of SalI for the construction of TrpE-His-CEACAM1-N or TrpE-His-CEACAM1-A1, or 10 units of BsiWI and 10 units of XhoI for the construction of pAT-TrpE-His-CEACAM1-N-m3C3d or pAT-TrpE-His-CEACAM1-A1-m3C3d to 20 μl [100 mM NaCl, 50 mM Tris-HCl, 10 mM MgCl₂, 1 mM DTT, pH 7.9] of the restriction enzyme reaction liquid, and then subjected to 1.2% agarose gel electrophoresis to purify the BsiWI-SalI fragment or the BsiWI-XhoI fragment of the CEACAM1 gene in the same manner as the above-described PCR product.

Next, 1 μg of DNA of pAT-trpE-His or pAT-trpE-His-m3C3d, which is an expression vector, is digested at 37° C. for 1 hour in a solution obtained by adding 10 units of BsiWI and 10 units of Sall for the construction of pAT-TrpE-His-CEACAM1-N or pAT-TrpE-His-CEACAM1-A1, and 10 units of BsiWI and 10 units of Xhol for the construction of pAT-TrpE-His-CEACAM1-N-m3C3d or pAT-TrpE-His-CEACAM1-A1-m3C3d to 20 μl of the restriction enzyme reaction liquid [100 mM NaCl, 50 mM Tris-HCl, 10 mM MgCl₂, 1 mM DTT, pH 7.9], and then subjected to 1.2% agarose gel electrophoresis, and, BsiWI-SalI or BsiWI-XhoI treated vectors were purified with Freeze 'N Squeeze™ DNA Gel Extraction Spin Columns (BIO-RAD).

An equal amount of DNA Ligation Kit <Mighty Mix> (Takara Bio Inc.) was added to a DNA solution in which the BsiWI-SalI or BsiWI-XhoI-treated vector DNA and the above-described restriction enzyme-treated CEACAM1 fragment were mixed so as to have the same weight, and the mixture was incubated at 16° C. for 30 minutes to perform a linking reaction. In order to obtain an expression plasmid, E. coli XL1-blue MRF (Stratagen) was transformed using this linking reaction liquid.

Sensitive E. coli strains used for transformation are produced by Mix & Go E. coli Transformation Kit & Buffer Set. Transformed E. coli was plated onto LB plates (1% tryptone, 0.5% NaCl, 1.5% agar) containing 100 μg/ml ampicillin and incubated at 37° C. overnight. Colonies of bacteria generated on the plate were taken with a sterile toothpick, transferred to 2YT medium containing 100 μg/ml ampicillin, and cultured at 37° C. overnight.

1.5 ml of the bacterial culture was centrifuged to collect the bacteria, and a mini-preparation of plasmid DNA was performed using Wizard® Plus SV Minipreps DNA Purification Systems (Promega). 1 μg of the obtained plasmid DNA was digested at 37° C. for 1 hour in a solution obtained by adding 10 units of BsiWI for the construction of TrpE-His-CEACAM1-N or A1 and 10 units of SalI, 10 units of BsiWI for the construction of TrpE-His-CEACAM1-N-m3C3d or Al-m3C3d and 10 units of XhoI to 20 μl of the restriction enzyme reaction liquid [100 mM NaCl, 50 mM Tris-HCl, 10 mM MgCl₂, 1 mM DTT, pH 7.9], then subjected to 1.2% agarose gel electrophoresis, and expression plasmids were selected that gave rise to a fragment of about 450 bp for pAT-TrpE-His-CEACAM1-N or pAT-TrpE-His-CEACAM1-N-m3C3d and about 400 bp for pAT-TrpE-His-CEACAM1-A1 or pAT-TrpE-His-CEACAM1-A1-m3C3d.

### (B) Expression and Purification of Antigen

E. coli transformed with pAT-TrpE-His-CEACAM1-N or pAT-TrpE-His-CEACAM1-N-m3C3d and pAT-TrpE-His-CEACAM1-A1 or pAT-TrpE-His-CEACAM1-A1-m3C3d were cultured at 37° C. overnight in LB medium containing 100 μg/ml ampicillin. This was inoculated into M9-CA containing 100 μg/ml ampicillin at a 1% concentration, and cultured at 37° C. overnight. After completion of the culture, the bacterial cells were collected by centrifugation, resuspended in 50 ml of Lysis solution [50 mM Tris-HCl (pH 8.5), 30 mM NaCl, 5 mM EDTA], 1 ml of lysozyme solution (10 mg/ml Lysozyme) was added, and treated at 37° C. for 1 hour. The suspension was sonicated (150 W, 2×90 seconds) to destroy the cells. Insoluble fractions were collected by centrifugation at 15,000 rpm for 30 min at 4° C. The insoluble fraction was resuspended in A solution [50 mM Tris-HCl (pH 8.5)] containing 1% of 50 ml NP40. The suspension was centrifuged at 15,000 rpm at 4° C. for 30 minutes to collect the insoluble fraction. The insoluble fraction was resuspended in A solution containing 50 ml of 2M urea. The suspension was centrifuged at 15000 rpm at 4° C. for 30 minutes to collect the insoluble fraction. The insoluble fraction was resuspended in 50 ml of solution A containing 6 M urea. The suspension was centrifuged at 15,000 rpm at 4° C. for 30 minutes to collect the soluble fraction.

Antigen proteins TrpE-His-CEACAM1-N, TrpE-His-CEACAM1-N-m3C3d, TrpE-His-CEACAM1-A1, and TrpE-His-CEACAM1-A1-m3C3d were purified from an antigen solution solubilized in a solution containing 6M urea by an ion exchange method using a S sepharose HP column (GE Healthcare) and an IMAC method using a HisTrap HP column (GE Healthcare).

### Example 2: Obtainment of Anti-CEACAM1 Monoclonal Antibody

### (A) Immunization

The antigen protein TrpE-His-CEACAM1-N-m3C3d or TrpE-His-CEACAM1-A1-m3C3d prepared by the above method was dissolved in 6M urea, then diluted in 10 mM phosphate buffer (pH 7.3) (PBS) containing 0.15 M NaCl so as to have a final concentration of 1.0 mg/ml, mixed with an equal amount of Freund's adjuvant, and subcutaneously administered to 4 to 6 week old BALB/c mice in an amount of 50 to 100 μg. Similar boosting was performed every 2 weeks, and 100 μg of an antigen protein dissolved in PBS was further administered intraperitoneally as a final immunization.

### (B) Cell Fusion

On the third day after final immunization, the spleen was aseptically extracted from this mouse, and the spleen was loosened into individual cells using tweezers and a cell strainer, and the spleen was suspended in RPMI1640 medium (without addition of serum). The cells precipitated by centrifugation (1000 rpm, 5 min, room temperature) were pipetted using 1 ml of Red Blood Cell Lysing Buffer (MERCK), and allowed to stand at room temperature for 1 minute. To the cells, 20 mL of RPMI1640 medium (without addition of serum) was added, and the cells were well suspended. The cells precipitated by centrifugation (1000 rpm, 5 min, room temperature) were suspended in RPMI1640 medium (without addition of serum). Myeloma cells in logarithmic growth phase (mouse myeloma cell line Sp2/0 Ag14) were collected from the culture flask, precipitated by centrifugation (1000 rpm, 5 min, room temperature), and suspended in RPMI1640 medium (without addition of serum). Myeloma cells and spleen cells were mixed at a cell number ratio of 1:1, and the mixture was dispensed so that the total cell number was 3.4×10⁷. The cell mixed liquid was centrifuged (1000 rpm, 5 min, room temperature), the supernatant was removed, and the mixture was washed twice with ECF buffer (0.3 M mannitol, 0.1 mM calcium chloride, 0.1 mM magnesium chloride solution). Finally, the mixed cell liquid suspended in 0.35 ml of ECF buffer was subjected to cell fusion using a cell fusion device ECFG21 (Nepa Gene Co., Ltd.) and an MS stand type chamber platinum electrode CUY497P2 (0.8 mL) under the conditions of a liquid volume of 0.35 ml, an AC voltage of 40 Vrms, an AC time of 10 sec, a DC voltage of 350 V, a pulse width of 30 usec, a pulse interval of 0.5 sec, a pulse number of 3 times, an attenuation rate of 10%, polarity switching+ (no switching), post fusion 7 sec, and an attenuated sine wave ON. Immediately after the fusion, the cells were suspended in RPMI1640 medium containing serum and allowed to stand for 10 minutes to recover the cell membrane. Thereafter, the cells were suspended in RPMI-1640 medium containing 10% fetal bovine serum and hypoxanthine, aminopterin, and thymidine (HAT), and seeded on a 96 well cell culture plate. After the hybridoma was cultured for about 10 days to grow only the hybridoma, the culture supernatant was collected and used for the screening described later.

(C) Screening of anti-CEACAM1 antibody-producing hybridomas

Hybridomas producing the antibody of interest were searched by ELISA method using the antigen proteins TrpE-His-CEACAM1-N and TrpE-His-CEACAM1-A1 described above. In order to select antibodies recognizing linear epitopes, various antigens were diluted to 1 μg/mL with TBS containing 1 mM TCEP and 6M urea. To a multimodule plate from Nunc Co., Ltd., 50 μL of the diluted antigen was added per well and allowed to stand at 4 to 8° C. overnight. After removing the antigen diluent, 100 μL of blocking liquid (0.1% casein, 1 mM EDTA, PBS) per well was added, and the plate was allowed to stand at room temperature for 1 hour to fix the antigen.

To each well, 50 μL of the culture supernatant diluted with the reaction liquid (0.1% casein, 1 mM EDTA, PBS) was added, and the plate was allowed to stand at room temperature for 1 hour. The wells were washed with PBS containing 0.05% Tween20, then 50 μL of horseradish peroxidase (HRP)-labeled anti-mouse IgG Fc-specific antibody was added, and the wells were allowed to stand at room temperature for 1 hour. The wells were washed with PBS containing 0.05% Tween20, and then 50 ML of a TMB solution was added to each well to induce color development. Thereafter, 2M H₂SO₄ was added to each well to stop the reaction, and the absorbance was measured at a wavelength of 450 nm.

The culture supernatant of the hybridoma reacted with both TrpE-His-CEACAM1-N and TrpE-His-CEACAM1-A1 was excluded because it is considered that the TrpE-His sequence or contaminant proteins derived from E. coli were recognized, and an antibody-producing hybridoma having a desired reaction specificity was obtained.

The hybridomas obtained from the mouse spleen immunized with the antigen protein TrpE-His-CEACAM1-N-m3C3d were designated as A3029, A3048, A3054, A3057, A3067, A3073, and A6056, and the hybridoma obtained from the mouse spleen immunized with the antigen protein TrpE-His-CEACAM1-A1-m3C3d was designated as A7004.

The epitope of the antibody produced by the obtained hybridoma is shown in Example 3, and hybridomas A3054 and A3067 that produce the antibody of the same epitope are 2 clones obtained from 500 hybridoma positive wells, hybridomas A3029, A3048, A3057, and A3073 that produce the antibody of the same epitope are 4 clones obtained from 500 hybridoma positive wells, A7004 is 1 clone obtained from 800 hybridoma positive wells, and A6056 is 1 clone obtained from 120 hybridoma positive wells.

### (D) Cloning of Hybridomas

The obtained hybridoma was subjected to single cloning by a limiting dilution method to establish an antibody-producing hybridoma.

### (E) Preparation of Anti-CEACAM1 Monoclonal Antibodies

The established mouse hybridoma was conditioned and cultured in a serum-free medium (Hybridoma-SEM, GIBCO). The cells were expanded to 50 mL in a T75 flask and transplanted into a culture bag (NIPRO CORPORATION) filled with 500 mL of serum-free culture medium when the cell density reached approximately 5E5 cells/ml. The cells were cultured for 2-4 weeks, and the culture supernatant was collected. The culture solution was applied to a column packed with protein G sepharose (GE Healthcare), and the bound antibody was eluted with a buffer at pH 3. The eluate was rapidly neutralized using 2M Tris (pH 8) and buffer exchanged into PBS using a desalting column. From 500 mL of the culture solution, 5 to 20 mg of anti-CEACAM1 monoclonal antibody could be obtained.

### Example 3: Epitope Analysis of Anti-CEACAM1 Monoclonal Antibodies

In order to analyze the epitope of the obtained anti-CEACAM1 monoclonal antibody, a deleted protein obtained by scraping the N-domain protein or the Al domain protein of CEACAM1 by 10 or 20 amino acids from the C-terminal and a deleted protein obtained by scraping the N-domain protein by 3 amino acids from the N-terminal were expressed in E. coli, and analyzed by dot blotting using the E. coli disrupted liquid.

### (A) Construction of Deleted Protein Expression Plasmid

Construction of the deleted protein expression plasmid was performed using KOD-Plus-Mutagenesis Kit (TOYOBO Co., Ltd.). As a template, pAT-GST-His-CEACAM1-N(Nfull) was used for construction of the N-domain deleted protein expression plasmid, and pAT-TrpE-His-CEACAM1-N-A1 (A1full) was used for construction of the A1 domain deleted protein expression plasmid. Primers used were set outside each region to be deleted. The amino acid sequences corresponding to the various deleted proteins are shown in Table 1. Inverse PCR, degradation of the template plasmid by DpnI, and phosphorylation and self-circularization of the PCR product were sequentially performed according to the manufacturer's instructions. E. coli XL1-blue MRF (Stratagen) was transformed using the self-cyclized PCR product. Transformed E. coli was plated onto LB plates (1% tryptone, 0.5% NaCl, 1.5% agar) containing 100 mg/ml ampicillin and incubated at 37° C. overnight. Colonies of bacteria generated on the plate were taken with a sterile toothpick, transferred to 2YT medium containing 100 μg/ml ampicillin, and cultured at 37° C. overnight. This was inoculated into M9-CA containing 100 μg/ml ampicillin at a 1% concentration, and cultured at 37° C. overnight. After completion of the culture, bacterial cells were collected by centrifugation and solubilized in 1×SDS-PAGE specimen buffer (62.5 mM Tris-HCl, 10% Glycerol, 2% SDS, 20 mM DTT) to prepare the E. coli disrupted liquids of respective deleted proteins. SDS-PAGE of the E. coli disrupted liquid was performed, and expression of a target protein was confirmed by Coomassie brilliant blue staining and an immunoblot using an anti-his-tag antibody.

TABLE 1
Amino acid sequences corresponding to various
deleted proteins

| Name of | |
|---|---|
| expressed | |
| protein | Amino acid sequence |
| N full | QLTTESMPFNVAEGKEVLLLVHNLPQQLFGYSWYKGERVDGNR |
| | QIVGYAIGTQQATPGPANSGRETIYPNASLLIQNVTONDTGFY |
| | TLQVIKSDLVNEEATGQFHVYP (SEQ ID NO: 24) |
| N-98 | QLTTESMPFNVAEGKEVLLLVHNLPQQLFGYSWYKGERVDGNR |
| | QIVGYAIGTQQATPGPANSGRETIYPNASLLIQNVTQNDTGFY |
| | TLQVIKSDLVNE (SEQ ID NO: 25) |
| N-88 | QLTTESMPFNVAEGKEVLLLVHNLPQQLFGYSWYKGERVDGNR |
| | QIVGYAIGTQQATPGPANSGRETIYPNASLLIQNVTQNDTGFY |
| | TL (SEQ ID NO: 26) |
| N-68 | QLTTESMPFNVAEGKEVLLLVHNLPQQLFGYSWYKGERVDGNR |
| | QIVGYAIGTQQATPGPANSGRETIY (SEQ ID NO: 27) |
| N-58 | QLTTESMPFNVAEGKEVLLLVHNLPQQLFGYSWYKGERVDGNR |
| | QIVGYAIGTQQATPG (SEQ ID NO: 28) |
| N-48 | QLTTESMPFNVAEGKEVLLLVHNLPQQLFGYSWYKGERVDGNR |
| | QIVGY (SEQ ID NO: 29) |
| N-28 | QLTTESMPFNVAEGKEVLLLVHNLPQQL (SEQ ID NO: 30) |
| N-18 | QLTTESMPFNVAEGKEVL (SEQ ID NO: 31) |
| N-8 | QLTTESMP (SEQ ID NO: 32) |
| N4-18 | TESMPENVAEGKEVL (SEQ ID NO: 33) |
| N7-18 | MPFNVAEGKEVL (SEQ ID NO: 34) |
| N10-18 | NVAEGKEVL (SEQ ID NO: 35) |
| A1 full | PKPSISSNNSNPVEDKDAVAFTCEPETQDTTYLWWINNQSLPV |
| | SPRLQLSNGNRTLTLLSVTRNDTGPYECEIQNPVSANRSDPVT |
| | LN (SEQ ID NO: 36) |
| A1-68 | PKPSISSNNSNPVEDKDAVAFTCEPETQDTTYLWWINNQSLPV |
| | SPRLQLSNGNRTLTLLSVTRNDTGP (SEQ ID NO: 37) |
| A1-48 | PKPSISSNNSNPVEDKDAVAFTCEPETQDTTYLWWINNQSLPV |
| | SPRLQ (SEQ ID NO: 38) |
| A1-38 | PKPSISSNNSNPVEDKDAVAFTCEPETQDTTYLWWINN (SEQ |
| | ID NO: 39) |
| A1-28 | PKPSISSNNSNPVEDKDAVAFTCEPETQ (SEQ ID NO: 40) |

GST-His is added to the N-terminus of the N-domain deleted protein, and TrpE-His-N is added to the N-terminus of the A1 domain deleted protein.

### (B) Dot Blotting

Each 1 μL of the various E. coli lysates described above was dotted on a nitrocellulose membrane and air-dried, and the nitrocellulose membrane was immersed in a transfer buffer (25 mM Tris, 192 mM Glycine, 20% MetOH) at room temperature for 10 minutes, and then blocking was performed using a reaction liquid (0.1% sodium caseinate, 1 mM EDTA, PBS). Each antibody diluted to 1 μg/mL with the reaction liquid was reacted with the dotted nitrocellulose membrane (dot membrane), and allowed to stand at room temperature for 1 hour. The dot membrane was washed with a washing liquid (0.05% Tween20, PBS), and reacted with horseradish peroxidase (HRP)-labeled anti-mouse IgGFc specific antibody diluted with the reaction liquid, and allowed to stand at room temperature for 1 hour.

The dot membrane was washed using a washing liquid, reacted with a luminescent substrate SuperSignal West Pico (Thermo scientific), allowed to stand at room temperature for 5 minutes, and then chemiluminescence was detected with a CCD imager.

### (C) Results

Epitope analysis by dot blotting showed that the amino acid sequence of TESMP (SEQ ID NO: 2) in the N domain of the A3054 and A3067 antibodies was an epitope. In the A3029, A3048, A3057, and A3073 antibodies, the amino acid sequence of PANSGRETIY (SEQ ID NO: 1) of the N domain was shown to be an epitope. The A6056 antibody was shown to be an epitope of the amino acid sequence of EATGOFHVYP (SEQ ID NO: 4) in the N domain. The A7004 antibody was shown to be an epitope of the amino acid sequence of DTTYLWWINN (SEQ ID NO: 3) in the A1 domain.

### Example 4: Cross-Reactivity Evaluation Using Recombinant Antigens

Using commercially available recombinant antigens CEACAM1 (R&D Systems), CEACAM3 (R&D Systems), CEACAM5 (R&D Systems), and CEACAM6 (R&D Systems), the cross-reactivity of the obtained anti-CEACAM1 monoclonal antibody (one type of antibody having the same epitope was selected) was evaluated.

(A) Antigen immobilized ELISA

The various antigens were diluted with TBS containing 1 mM TCEP and 6M urea to 1 μg/mL. To a multimodule plate from Nunc Co., Ltd., 50 μL of the diluted antigen was added per well and allowed to stand at room temperature overnight. The antigen diluent was removed, and the plate was washed with PBS, then 100 μL of a blocking liquid (0.1% sodium caseinate, 150 mM NaCl, 1 mM EDTA, PBS) per well was added, and the plate was allowed to stand at room temperature for 1 hour.

The blocking liquid was removed, 50 μL of each of various antibodies diluted with a reaction liquid (0.1% sodium caseinate, 150 mM NaCl, 1 mM EDTA, PBS) was added to each well, and the plate was allowed to stand at room temperature for 1 hour. The wells were washed with PBS containing 0.05% Tween20, then 50 μL of horseradish peroxidase (HRP)-labeled anti-mouse IgG Fc-specific antibody was added, and the wells were allowed to stand at room temperature for 1 hour. The wells were washed with PBS containing 0.05% Tween20, and then 50 μL of a TMB solution was added to each well to induce color development. Thereafter, 2M H₂SO₄ was added to each well to stop the reaction, and the absorbance was measured at a wavelength of 450 nm.

### (B) Results

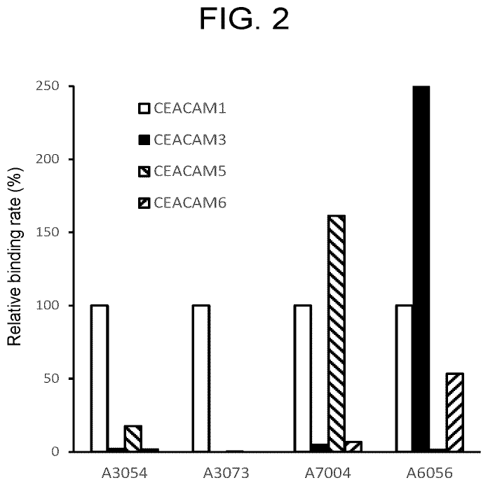

The results of the cross-reactivity evaluation of the CEACAM1 antibody are illustrated in FIG. 2. Reactivity to CEACAM3, CEACAM5 and CEACAM6 was expressed as relative values to CEACAM1 (relative binding rate (%)). The A3054 antibody showed about 18% cross-reactivity to CEACAM5, but less than 5% cross-reactivity to CEACAM3 and CEACAM6. The A3073 antibody shows no cross-reactivity to either CEACAM3, CEACAM5 or CEACAM6, with the highest CEACAM1 specificity. The A6056 antibody did not show cross-reactivity to CEACAM5, but showed cross-reactivity to CEACAM6 of approximately 50% and to CEACAM3 of 250%. The A7004 antibody showed about 5% cross-reactivity to CEACAM3 and CEACAM6, and 150% cross-reactivity to CEACAM5.

### Example 5: Sequence Comparison with CEACAM Family

When comparing the epitope of each antibody with the amino acid sequence alignment of the CEACAM family (FIG. 3), it can be seen that the A3054 antibody and the A3073 antibody recognize a sequence including amino acids (threonine residue (T) in TESMP (SEQ ID NO: 2) and asparagine residue (N) in PANSGRETIY (SEQ ID NO: 1), respectively.) unique to CEACAM1 (indicated by black frames in FIG. 3). From the evaluation results of cross-reactivity, since these two antibodies have low cross-reactivity to CEACAM5 and CEACAM6, these amino acids unique to CEACAM1 are considered to be hot spots of epitopes. In the evaluation result of cross-reactivity, A6056 antibody showed a moderate cross-reaction to CEACAM6, and no cross-reaction to CEACAM5, so that the histidine residue (H) in EATGQFHVYP (SEQ ID NO: 4) is considered to be a hot spot of an epitope (indicated by black frames in FIG. 3). Since this sequence is also present in CEACAM3 and CEACAM4, the A6056 antibody may cross-react with these. In the evaluation result of cross-reactivity, since the A7004 antibody strongly cross-reacted with CEACAM5 and did not react with CEACAM6, the aspartic acid residue (D) in DTTYLWWINN (SEQ ID NO: 3) is considered to be a hot spot of an epitope (indicated by black frames in FIG. 3). Since only CEACAM1 and CEACAM5 have this sequence, it is considered that this sequence does not cross-react with other CEACAM families.

### Example 6: Cross-Reactivity Evaluation Using Peptide

Cross-reactivity evaluation using a synthetic peptide was performed on the A3054 and A3067 antibodies and the A3029, A3048, A3067, and A3073 antibodies considered to recognize the CEACAM1 specific sequence.

### (A) Peptide Immobilized ELISA

The various synthetic peptides were diluted with TBS containing 1 mM TCEP and 6M urea to 20 μg/mL. To a multimodule plate from Nunc Co., Ltd., 50 μL of the diluted antigen was added per well and allowed to stand at room temperature overnight. The antigen diluent was removed, and the plate was washed with PBS, then 100 μL of a blocking liquid (0.1% sodium caseinate, 150 mM NaCl, 1 mM EDTA, PBS) per well was added, and the plate was allowed to stand at room temperature for 1 hour.

The blocking liquid was removed, 50 μL of each of various antibodies diluted with a reaction liquid (0.1% sodium caseinate, 150 mM NaCl, 1 mM EDTA, PBS) was added to each well, and the plate was allowed to stand at room temperature for 1 hour. The wells were washed with PBS containing 0.05% Tween20, then 50 μL of horseradish peroxidase (HRP)-labeled anti-mouse IgG Fc-specific antibody was added, and the wells were allowed to stand at room temperature for 1 hour. The wells were washed with PBS containing 0.05% Tween20, and then 50 μL of a TMB solution was added to each well to induce color development. Thereafter, 2M H₂SO₄ was added to each well to stop the reaction, and the absorbance was measured at a wavelength of 450 nm.

### (B) Results

Tables 2 and 3 show the cross-reactivity of each antibody as a ratio (%) to the sequence of CEACAM1. The A3054 and A3067 antibodies showed cross-reactivity to CEACAM3, CEACAM5, and CEACAM6. A3073, A3029, A3048, A3057 did not show cross-reactivity to sequences of any CEACAM family member, indicating high CEACAM1 specificity.

TABLE 2
Cross-reactivity of A3054 and A3067 antibodies

| Name of | | | |
|---|---|---|---|
| peptide | Amino acid sequence | A3054 | A3067 |
| CEACAM1 | QLTTESMPEN (SEQ ID NO: 41) | 100% | 100% |
| CEACAM3 | KLTIESMPLS (SEQ ID NO: 42) | 79% | 81% |
| CEACAM4 | QFTIEALPSS (SEQ ID NO: 43) | 0% | 0% |
| CEACAM5, 6 | KLTIESTPEN (SEQ ID NO: 44) | 97% | 103% |
| CEACAM7 | QTNIDVVPEN (SEQ ID NO: 45) | 0% | 0% |
| CEACAM8 | QLTIEAVPSN (SEQ ID NO: 46) | 0% | 0% |

% CEACAM1

TABLE 3
Cross-reactivity of A3029, A3048, A3057 and A3073
antibodies

| Name of | | | | | |
|---|---|---|---|---|---|
| peptide | Amino acid sequence | A3029 | A3048 | A3057 | A3073 |
| CEACAM1 | QATPGPANSGRETIY | 100% | 100% | 100% | 100% |
| | (SEQ ID NO: 47) | | | | |
| CEACAM3 | QATPGAAYSGRETIY | 0% | 0% | 0% | 0% |
| | (SEQ ID NO: 48) | | | | |
| CEACAM4 | ANIPGAAYSGRETVY | 0% | 0% | 0% | 0% |
| | (SEQ ID NO: 49) | | | | |
| CEACAM5 | QATPGPAYSGREIIY | 0% | 0% | 1% | 0% |
| | (SEQ ID NO: 50) | | | | |
| CEACAM6 | QATPGPAYSGRETIY | 0% | 0% | 0% | 0% |
| | (SEQ ID NO: 51) | | | | |
| CEACAM7 | ENAPGPAHNGRETIY | 0% | 0% | 0% | 0% |
| | (SEQ ID NO: 52) | | | | |
| CEACAM8 | QITPGPAYSNRETIY | 0% | 0% | 0% | 0% |
| | (SEQ ID NO: 53) | | | | |

% CEACAM1

### Example 7: Construction of CEACAM1 Sandwich Measurement System

### (A) Preparation of Biotin-Labeled Antibody

The buffer of the antibody solution was replaced with PBS using a desalting column, and the antibody concentration was adjusted to 0.5 mg/mL. Sulfo-NHS-LC-Biotin (ThermoScientific) was added to this antibody solution so as to be about 70 μM, and the mixture was reacted at room temperature for 90 minutes. Thereto was added 1/20 volume of 1 M Tris (pH 7) to stop the reaction, thereby obtaining a biotin-labeled form.

### (B) Sandwich ELISA Antibody Combination

Various antibodies were diluted with PBS to 5 or 10 μg/mL. To a multimodule plate from Nunc Co., Ltd., 50 μL of the diluted antibody was added per well and allowed to stand at 4 to 8° C. overnight. The antibody diluent was removed, and the plate was washed with PBS, then 100 μL of a blocking liquid (0.1% sodium caseinate, 150 mM NaCl, 1 mM EDTA, PBS) per well was added, and the plate was allowed to stand at room temperature for 1 hour to prepare an antibody immobilized plate. 50 μL of specimen obtained by diluting commercially available CEACAM1 antigen (R&D Systems) with Lumipulse (registered trademark) specimen diluent (Fujirebio Inc.) to 1 or 0 μg/mL was mixed with 50 μL of a denaturing solution (10% SDS, 3.75% EDTA2Na, 10 mM TCEP), shaken at 1000 rpm, and heated at 80° C. for 10 minutes to prepare a denaturation treatment specimen. 20 μL of the denaturation treatment specimen was mixed with 100 μL of a reaction liquid (100 mM Tris-HCl, 200 mM NaCl, 20 mM EDTA 3Na, 0.1% ProClin 300, 2% BSA, pH 7.5), and 50 μL of the mixture was added to the well of the antibody immobilized plate from which the blocking liquid had been removed and allowed to stand at room temperature for 1 hour. The well was washed with PBS containing 0.05% Tween20, then a biotin-labeled antibody diluted to 1 μg/mL with a labeled form diluent (50 mM MES, 100 mM NaCl, 0.3 mM ZnCl₂, 1 mM MgCl₂, 2% BSA, 0.10% NaN₃, pH 6.8) was added to the well, and the well was allowed to stand at room temperature for 1 hour. The well was washed with PBS containing 0.05% Tween20, then alkaline phosphatase (ALP)-labeled streptavidin diluted with a diluent of the labeled form was added to the well, and the well was allowed to stand at room temperature for 30 minutes. The well was washed with PBS containing 0.05% Tween20, then a Lumipulse substrate liquid (Fujirebio Inc.) was added, and the amount of chemiluminescence by the enzyme reaction was measured.

The results of studies on a combination of an immobilized antibody and a labeled antibody in sandwich ELISA are shown in Table 4. The θ count value is a difference obtained by subtracting the measured value of 0 μg/mL from the measured value of 1 μg/mL of CEACAM1 antigen. When the A3073 antibody having the highest CEACAM1 specificity was used for immobilization and the A7004 antibody or the A6056 antibody was used as a labeled form, high reactivity was exhibited.

TABLE 4
Combination of immobilized antibody and
labeled antibody in sandwich ELISA

| | | Immobilized antibody | | | |
|---|---|---|---|---|---|
| Δ0 Count Value | | A3054 | A3073 | A7004 | A6056 |
| Biotin-labeled | A3054 | | 78170 | 1010 | 560 |
| antibody | A3073 | 72890 | | 26140 | 9550 |
| | A7004 | 3870 | 145160 | | 2550 |
| | A6056 | 14520 | 161880 | 4320 | |

### (C) Immobilized Antibody Combinations

The effect of combining the A3054 antibody (or A3067 antibody) and the A3073 antibody (or A3029, A3048, A3057) as immobilized antibodies was examined. The measurement method followed the “(B) sandwich ELISA antibody combination” described above. As an immobilized condition, they were diluted by using PBS to 5 or 10 μg/mL in the case of a single immobilization, and they were mixed so as to be 5 μg/mL in the case of a co-immobilization. A specimen obtained by diluting a commercially available CEACAM1 antigen (R&D Systems) with a Lumipulse specimen diluent (Fujirebio Inc.) to 1 or 0 μg/mL was measured.

The results of the immobilized antibody combination are shown in Table 5. The Δ0 count value is a difference obtained by subtracting the measured value of 0 μg/mL from each of the measured values of 1 μg/mL of the CEACAM1 antigen. The co-immobilization showed higher reactivity than the single immobilization of each of the A3054 antibody (or the A3067 antibody) and the A3073 antibody (or A3029, A3048, and A3057). Since this value is larger than the sum of the individual immobilization, it is considered that a synergistic effect was exhibited in the CEACAM1 binding force.

TABLE 5
Combinations of immobilized antibodies

| | |
|---|---|
| Immobilization | Δ0 Count Value |

| | | | |
|---|---|---|---|
| Immobilized antibody | conc. (μg/mL) | A6056 | A7004 |

| A3054 | | 5 | 380 | 670 |
|---|---|---|---|---|
| | | 10 | 540 | 1760 |
| A3067 | | 5 | 270 | 2860 |
| | | 10 | 270 | 4920 |
| A3029 | | 5 | 14695 | 46630 |
| | | 10 | 19800 | 55705 |
| A3048 | | 5 | 18735 | 61915 |
| | | 10 | 23710 | 78160 |
| A3057 | | 5 | 10710 | 42610 |
| | | 10 | 14950 | 53110 |
| A3073 | | 5 | 11530 | 49640 |
| | | 10 | 12990 | 57395 |
| A3054+ | A3029 | 5 + 5 | 66713 | 173820 |
| | A3048 | 5 + 5 | 55453 | 167410 |
| | A3057 | 5 + 5 | 57167 | 168395 |
| | A3073 | 5 + 5 | 34545 | 145560 |
| A3067+ | A3029 | 5 + 5 | 42240 | 146645 |
| | A3048 | 5 + 5 | 35333 | 143515 |
| | A3057 | 5 + 5 | 36300 | 143410 |
| | A3073 | 5 + 5 | 22005 | 121520 |

### (D) Cross-Reactivity Evaluation in Sandwich Measurement System

Cross-reactivity was confirmed when the A3054 antibody (or A3067 antibody) and the A3073 antibody (or A3029, A3048, and A3057) were combined as immobilized antibodies. The measurement method followed the “(B) sandwich ELISA antibody combination” described above. As a immobilized condition, PBS was used, and the A3054 antibody (or the A3067 antibody) and the A3073 antibody (or A3029, A3048, A3057) were mixed so that each was 5 μg/mL. Specimens obtained by diluting commercially available CEACAM1 antigen (R&D Systems), CEACAM3 antigen (R&D Systems), CEACAM5 antigen (R&D Systems), and CEACAM6 antigen (R&D Systems) to 1 or 0 μg/mL with a Lumipulse specimen diluent (Fujirebio Inc.) were measured.

The results of the cross-reactivity evaluation in the sandwich measurement system are shown in Table 6. The Δ0 count value is a difference obtained by subtracting the measured value of 0 μg/mL from the measured value of 1 μg/mL of CEACAM1 antigen. No significant cross-reaction to CEACAM3, CEACAM5 and CEACAM6 was shown in any immobilized antibody combination. It has been shown that the A3054 antibody with cross-reactivity (or A3067 antibody) does not affect the CEACAM1 specificity of the A3073 antibody (or A3029, A3048, and A3057).

TABLE 6
Cross-reactivity in sandwich measurement system

| Labeled | Immobilized antibody | | Δ0 Count Value | | | | vs. CEACAM1 (%) | | |
|---|---|---|---|---|---|---|---|---|---|
| antibody | combination | | CEACAM1 | CEACAM3 | CEACAM5 | CEACAM6 | CEACAM3 | CEACAM5 | CEACAM6 |
| A6056 | A3054 | A3029 | 66713 | 183 | 113 | 483 | 0.3% | 0.2% | 0.7% |
| | | A3048 | 55453 | 163 | 0 | 243 | 0.3% | 0.0% | 0.4% |
| | | A3057 | 57167 | 87 | 217 | 737 | 0.2% | 0.4% | 1.3% |
| | | A3073 | 34545 | 265 | 215 | 285 | 0.8% | 0.6% | 0.8% |
| | A3067 | A3029 | 42240 | 580 | 0 | 760 | 1.4% | 0.0% | 1.8% |
| | | A3048 | 35333 | 433 | 513 | 343 | 1.2% | 1.5% | 1.0% |
| | | A3057 | 36300 | 170 | 0 | 160 | 0.5% | 0.0% | 0.4% |
| | | A3073 | 22005 | 0 | 185 | 335 | 0.0% | 0.8% | 1.5% |
| A7004 | A3054 | A3029 | 173820 | 0 | 690 | 50 | 0.0% | 0.4% | 0.0% |
| | | A3048 | 167410 | 0 | 360 | 0 | 0.0% | 0.2% | 0.0% |
| | | A3057 | 168395 | 0 | 545 | 0 | 0.0% | 0.3% | 0.0% |
| | | A3073 | 145560 | 0 | 320 | 0 | 0.0% | 0.2% | 0.0% |
| | A3067 | A3029 | 146645 | 0 | 405 | 0 | 0.0% | 0.3% | 0.0% |
| | | A3048 | 143515 | 0 | 295 | 0 | 0.0% | 0.2% | 0.0% |
| | | A3057 | 143410 | 30 | 580 | 400 | 0.0% | 0.4% | 0.3% |
| | | A3073 | 121520 | 30 | 670 | 300 | 0.0% | 0.6% | 0.2% |

### Example 8: Evaluation of CEACAM1 Measurement System Using Fully Automated Chemiluminescent Enzyme Immunoassay System

### (A) Preparation of Anti-CEACAM1 Antibody-Bound Ferrite Particles

Anti-CEACAM1 antibodies (A3054 and A3073) were added to the magnetic particles in a 10 mM MES buffer (pH 5.0) to obtain a suspension containing 0.04 mg/mL anti-CEACAM1 antibodies (A3054 and A3073) and 5 mg/mL magnetic particles. The suspension was incubated at 25° C. for 1 hour with gentle stirring to immobilize the anti-CEACAM1 antibodies (A3054 and A3073) on the magnetic particles. Thereafter, the magnetic particles were magnetically collected with a magnet, and the magnetic particles were washed with a washing liquid (50 mM Tris buffer, 150 mM NaCl, 2.0% BSA, pH 7.2) to obtain anti-CEACAM1 antibody (A3054 and A3073) immobilized particles.

### (B) Preparation of Alkaline Phosphatase (ALP)-Labeled Anti-CEACAM1 Antibody

The desalted alkaline phosphatase and N-(4-maleimidobutyryloxy)-succinimide (GMBS) (final concentration: 0.3 mg/mL) were mixed in a 0.1 M phosphate buffer (pH 7.0), and the mixture was allowed to stand at 30° C. for 1 hour to perform maleimidation. After desalting using a coupling buffer, Fab′-converted anti-CEACAM1 monoclonal antibody A7004 and maleimidized alkaline phosphatase were mixed at a molar ratio of 1:1, and the mixture was allowed to stand at 25° C. for 30 minutes to perform coupling. The mixed liquid of the reacted antibody and ALP was concentrated, and gel-filtration purification was performed using an ALP buffer (1 mM MgCl₂, 0.1 mM ZnCl₂, 0.1% NaN₃, 0.15 M NaCl, 0.1 M MES, pH 6.8) to obtain an ALP-labeled anti-CEACAM1 antibody.

### (C) CEACAM1 Measurement Method

CEACAM1 was measured using a fully automated chemiluminescent enzyme immunoassay system according to the following method. 50 μL of the specimen and 50 μL of a denaturation treatment liquid (10% SDS, 3.75% EDTA2Na, 10 mM TCEP) were mixed, and the mixture was shaken at 1000 rpm and heated at 80° C. for 10 minutes to prepare a denaturation treatment specimen. Among them, 50 μL was mixed with 50 μL of a magnetic particle liquid (100 mM Tris-HCl, 200 mM NaCl, 20 mM EDTA 3Na, 0.1% ProClin 300, 2% BSA, pH 7.5) containing magnetic particles in which the anti-CEACAM1 antibodies A3054 and A3073 were co-bound, and the mixture was reacted for 8 minutes. The magnetic particles were magnetically collected and washed to remove any components unbound to the magnetic particles, and 50 μL of ALP-labeled A7004 antibody diluted to 0.2 μg/mL with labeled form diluent (50 mM MES, 100 mM NaCl, 0.3 mM ZnCl₂, 1 mM MgCl₂, 2% BSA, 0.10% NaN3, pH 6.8) was added and the mixture allowed to react for 8 minutes. The magnetic particles were magnetically collected and washed to remove components unbound to the magnetic particles, and 200 μL of a substrate liquid (Lumipulse substrate liquid, manufactured by Fujirebio Inc.) containing AMPDD was added. The amount of luminescence due to the enzyme reaction was counted, and the CEACAM1 value in the specimen was calculated from the count using a calibration curve. A calibration curve was prepared based on the amount of luminescence obtained for each standard liquid by measuring CEACAM1 (R&D Systems) standard liquids corresponding to CEACAM1 amounts of 0, 1.6, 8, 40, 200, and 1000 ng/mL in the same manner as for the specimens. All the steps after the specimen denaturation treatment of this example were performed using an automated analyzing device Lumipulse L2400 (registered trademark, manufactured by Fujirebio Inc.).

### (D) Measurement of Disease Specimen

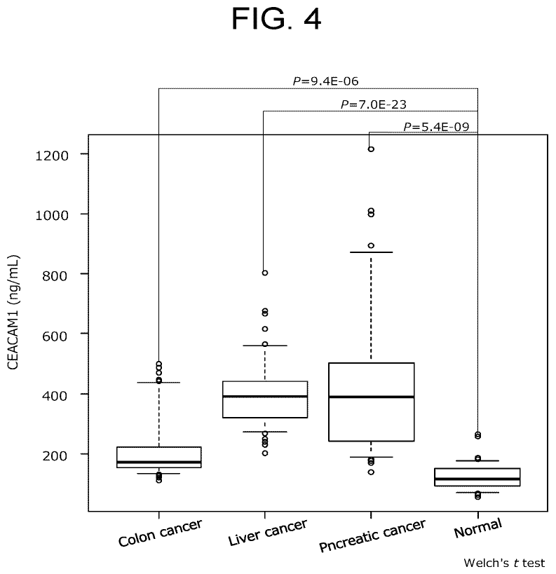

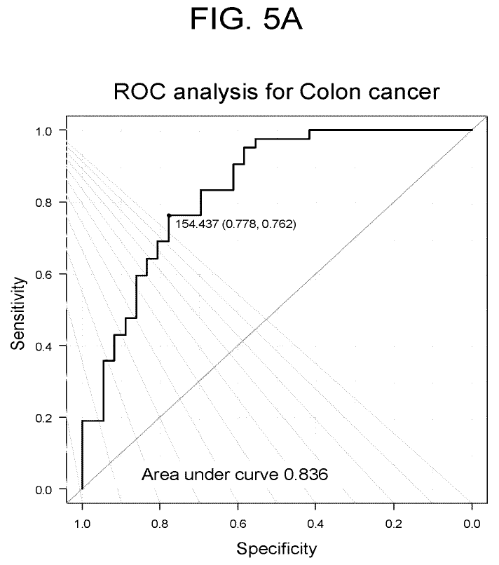

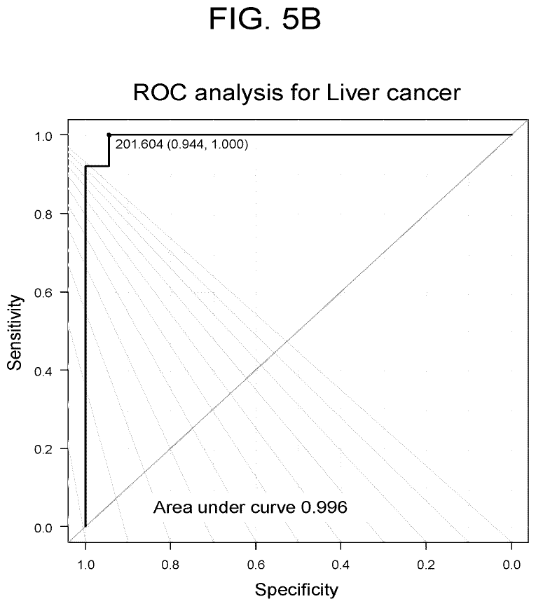

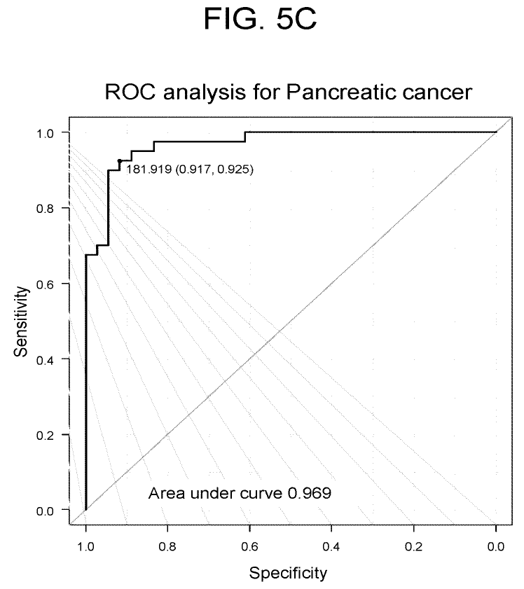

A colon cancer specimen (purchased from TRINA BIOREACTIVES AG) (n=42), a liver cancer specimen (purchased from TRINA BIOREACTIVES AG) (n=50), a pancreatic cancer specimen (purchased from TRINA BIOREACTIVES AG) (n=40), and a healthy human specimen (n=36) were measured by the CEACAM1 measurement method using the fully automated chemiluminescent enzyme immunoassay system described above. FIG. 4 illustrates the view of these blood concentration distributions, and FIG. 5 illustrates a receiver operating characteristic (ROC) analysis view in each cancer specimen. The CEACAM1 concentration of the healthy human specimen is 125.7±49.4 ng/mL on average, indicating a relatively stable value. The CEACAM1 average concentrations of the colon cancer specimen, the liver cancer specimen, and the pancreatic cancer specimen were 216.1±108.4 ng/ml, 399.9±119.3 ng/ml, and 430.9±259.4 ng/ml, respectively, which were significantly higher than those of the healthy human specimen. As a result of ROC analysis, in the colon cancer specimen, the area under curve was 0.836, and the specificity and the sensitivity when the cut-off value was 154.4 ng/mL were 0.778 and 0.762, respectively. In the liver cancer specimens, the area under curve was 0.996, and the specificity and sensitivity were 0.994 and 1.0, respectively, when the cut-off value was 201.6 ng/ml. In the pancreatic cancer specimens, the area under curve was 0.969, and the specificity and sensitivity were 0.917 and 0.925, respectively, when the cut-off value was 181.9 ng/mL.

### (E) Evaluation of CEACAM1 Specificity by CEA Absorption

In order to evaluate the specificity of the CEACAM1 measurement system, CEACAM1 measured values of a specimen [absorbed (+)] in which CEA was absorbed and an untreated specimen [absorbed (−)] were compared using an anti-CEA monoclonal antibody-bound particle liquid of a Lumipulse Presto CEA measurement reagent (manufactured by Fujirebio Inc.), and it was confirmed that the specimens were not affected by coexisting CEA.

50 μL of the anti-CEA monoclonal antibody-bound particle solution concentrated 2 times and 50 μL of a specimen were mixed, and the mixture was shaken at 1000 rpm and reacted at 37° C. for 30 minutes. The magnetic particles were magnetically collected, and the supernatant was collected to prepare a specimen [absorbed (+)] absorbing CEA. In addition, 50 μL of the particle-free particle diluent and 50 μL of the specimen were mixed, and the mixture was shaken at 1000 rpm and reacted at 37° C. for 30 minutes to prepare an untreated specimen [absorption (−)]. CEACAM1 measurement and CEA measurement were performed using these specimens. CEACAM1 measurement was performed according to the CEACAM1 measurement method using the fully automated chemiluminescent enzyme immunoassay system described above, and CEA measurement was performed according to the package insert of the Lumipulse Presto CEA measurement reagent (manufactured by Fujirebio Inc.) using the automated analyzing device LUMIPULSE L2400 (manufactured by Fujirebio Inc.).

Table 7 shows the evaluation results of CEACAM1 specificity by CEA absorption. In specimen 1 and specimen 2 in which the CEA measured value was more than 100 ng/mL, there was almost no change in the CEACAM1 measured value although the absorption (+) was a condition capable of absorbing about 908 of CEA. This showed that the constructed CEACAM1 measurement system specifically detected CEACAM1 without being affected by CEA.

TABLE 7
Evaluation of CEACAM1 specificity by CEA absorption

| | CEACAM1 measurement | | | CEA measurement | | |
|---|---|---|---|---|---|---|
| | Absorption | Absorption | | Absorption | Absorption | |
| | (+) | (−) | Absorption | (+) | (−) | Absorption |
| Specimen | ng/mL | ng/mL | (+)/(−) | ng/mL | ng/mL | (+)/(−) |
| 1 | 80.4 | 81.8 | 0.98 | 36.4 | 371.5 | 0.10 |
| 2 | 145.3 | 138.9 | 1.05 | 18.6 | 137.7 | 0.14 |
| 3 | 58.3 | 63.0 | 0.93 | 0.2 | 0.5 | 0.43 |
| 4 | 37.4 | 37.1 | 1.01 | 0.3 | 0.7 | 0.41 |

### Example 9: Study of Method for Exposing Epitope Of Anti-CEACAM1 Antibody

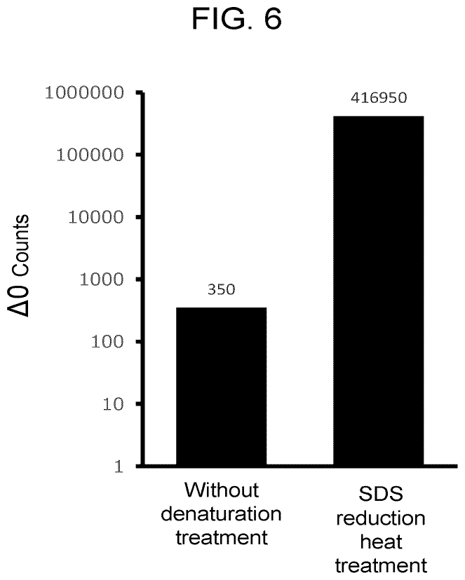

Since the anti-CEACAM1 antibody obtained in the present invention recognizes a linear epitope, an antigen denaturation treatment step is essential in order to enhance antigen-antibody reaction. In the examples described above, SDS reduction heat treatment was used as a denaturation treatment method of the antigen. As illustrated in FIG. 6, the epitope is exposed by subjecting the antigen to SDS reduction heat treatment, and the CEACAM1 specific signal is increased by about 1000 times. As another denaturation treatment method, a denaturation treatment with an alkali was examined.

### (A) Study of Alkali Treatment

A3054 and A3073 were each diluted with PBS to 5 μg/mL. To a multimodule plate from Nunc Co., Ltd., 50 μL of the diluted antibody was added per well and allowed to stand at 4 to 8° C. overnight. The antibody liquid in the well was removed, and the plate was washed with PBS, then 100 μL of a blocking liquid (0.1% sodium caseinate, 150 mM NaCl, 1 mM EDTA, PBS) per well was added, and the plate was allowed to stand at room temperature for 1 hour to prepare an antibody immobilized plate. 50 μL of a specimen obtained by diluting a commercially available CEACAM1 antigen (R&D Systems) with a Lumipulse specimen diluent (Fujirebio Inc.) to 1000 or 0 ng/mL was mixed with 50 μL of a denaturation treatment liquid (0 to 0.5 M NaOH), and the mixture was incubated at 37° C. for 7 minutes to prepare a denaturation treatment specimen. 50 μL of the denaturation treatment specimen was mixed with 50 μL of a reaction liquid (1.5 M Tris-HCl, 100 mM NaCl, 10 mM EDTA 3Na, 0.1% ProClin 300, 5% BSA, pH 7.5), and 50 μL of the mixture was added to the well of the antibody immobilized plate from which the blocking liquid had been removed and allowed to stand at room temperature for 1 hour. The well was washed with PBS containing 0.05% Tween20, then an ALP-labeled A7004 antibody diluted to 1 μg/mL with a labeled form diluent (50 mM MES, 100 mM NaCl, 0.3 mM ZnCl₂, 1 mM MgCl₂, 2% BSA, 0.10% NaN₃, pH 6.8) was added to the well, and the well was allowed to stand at room temperature for 1 hour. The well was washed with PBS containing 0.05% Tween20, then a Lumipulse substrate liquid (Fujirebio Inc.) was added, and the amount of chemiluminescence by the enzyme reaction was measured.

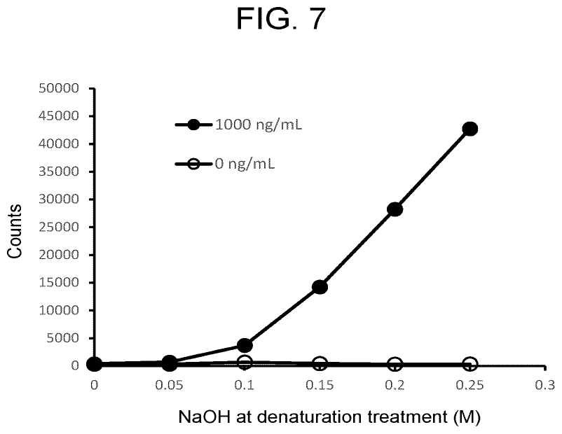

FIG. 7 illustrates the examination results of the denaturation treatment with alkali. In the alkali treatment with NaOH, the CEACAM1-specific signal increases in a concentration-dependent manner. From this result, it was shown that not only SDS reduction heat treatment but also alkali treatment was effective for exposure of the epitope.

### (B) Optimization of Alkali Treatment NaOH Concentration

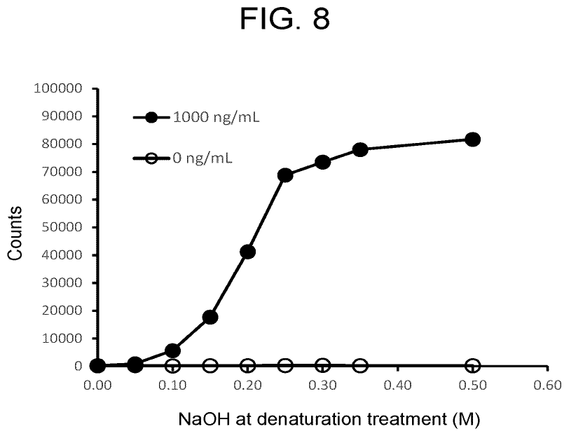

According to the measurement method described above, the NaOH concentration of the denaturation treatment liquid was optimized at 0 to 1 M. Results are illustrated in FIG. 8. It was shown that the CEACAM1-specific signal was increased in a concentration-dependent manner by the NaOH treatment, and the effect of the alkali treatment almost reached a plateau at a concentration of 0.25 M or more at the time of the denaturation treatment.

### Example 10: Measurement of CEACAM1 by Alkali Denaturation Treatment

### (A) Measurement of CEACAM1 Using a Fully Automated Chemiluminescent Enzyme Immunoassay System

25 μL of the specimen and 25 μL of a denaturation treatment liquid (0.8 M NaOH, 0.5% Brij35 (polyoxyethylene 23 lauryl ether)) were mixed, and a denaturation treatment was performed at 37° C. for 6.5 minutes. Thereto was added 50 μL of a neutralizing liquid (1.5 M Tris, 10 mM EDTA 3Na, 2% BSA, 0.1% ProClin 300, pH 7.5) for neutralization, then the mixture was mixed with 50 μL of a magnetic particle liquid (1.5 M Tris-HCl, 20 mM EDTA 3Na, 0.1% ProClin 300, 2% BSA, pH 7.5) containing magnetic particles in which the anti-CEACAM1 antibodies A3054 and A3073 were co-bound, and reacted for 8 minutes. The magnetic particles were magnetically collected and washed to remove unbound components from the magnetic particles, and 50 μL of ALP-labeled A7004 antibody diluted to 0.2 mg/ml with a labeled form diluent (50 mM MES, 100 mM NaCl, 0.3 mM ZnCl₂, 1 mM MgCl₂, 2% BSA, 0.10% NaN₃, pH 6.8) was added and reacted for 8 minutes. The magnetic particles were magnetically collected and washed to remove components unbound to the magnetic particles, and 200 μL of a substrate liquid (Lumipulse substrate liquid, manufactured by Fujirebio Inc.) containing AMPDD was added. The amount of luminescence due to the enzyme reaction was counted, and the CEACAM1 value in the specimen was calculated from the count using a calibration curve. For the calibration curve, CEACAM1 (R&D Systems) standard liquids corresponding to CEACAM1 amounts of 0, 8, 40, 200, and 1000 ng/ml were measured in the same manner as the specimen, and the calibration curve was prepared based on the amount of luminescence obtained for each standard liquid. All the processes of the present example were performed using an automated analyzing device LUMIPULSE L2400 (manufactured by Fujirebio Inc.).

### (B) Measurement of Disease Specimen

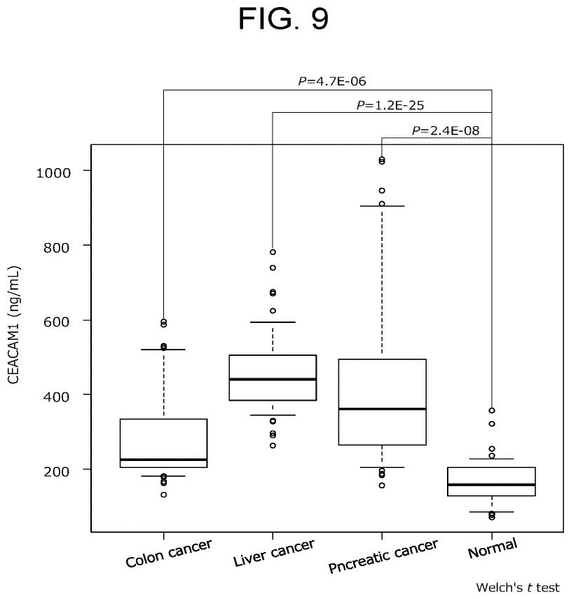

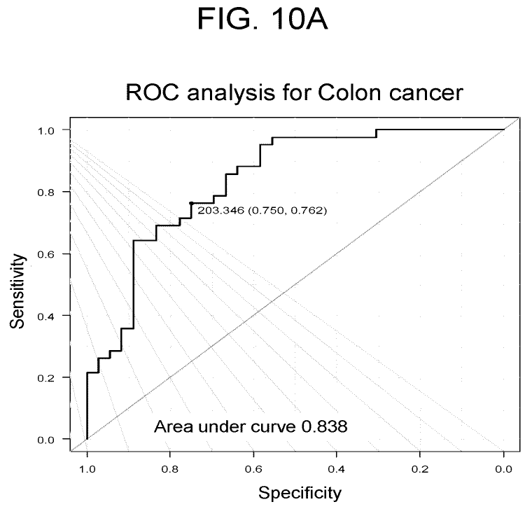

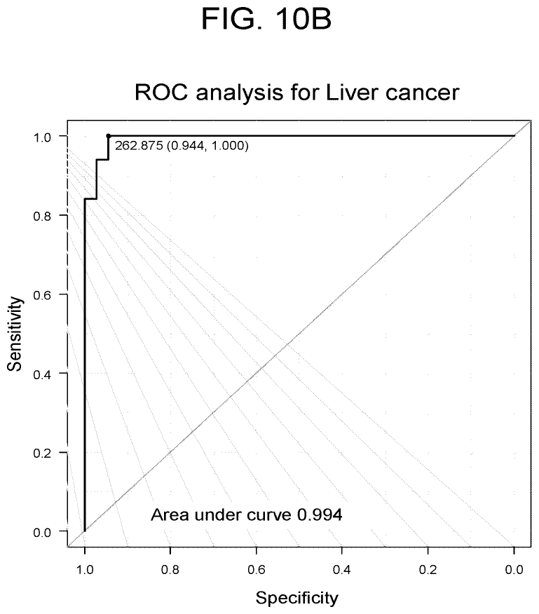

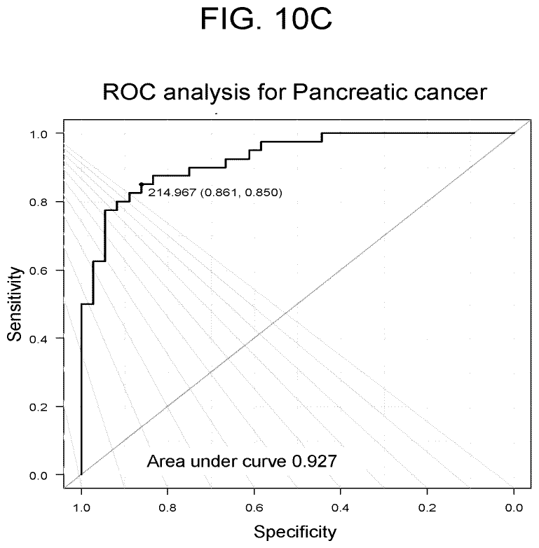

A colon cancer specimen (purchased from TRINA BIOREACTIVES AG) (n=42), a liver cancer specimen (purchased from TRINA BIOREACTIVES AG) (n=50), a pancreatic cancer specimen (purchased from TRINA BIOREACTIVES AG) (n=41), and a healthy human specimen (n=36) were measured by the CEACAM1 measurement method using the fully automated chemiluminescent enzyme immunoassay system described above. FIG. 9 illustrates the view of these blood concentration distributions, and FIG. 10 illustrates a receiver operating characteristic (ROC) analysis view in each cancer specimen. The CEACAM1 concentration of the healthy human specimen is 166.3±64.9 ng/ml on average, indicating a relatively stable value. The CEACAM1 average concentrations of the colon cancer specimen, the liver cancer specimen, and the pancreatic cancer specimen were 276.7±124.7 ng/ml, 458.716±111.2 ng/ml, and 424.3±237.7 ng/ml, respectively, which were significantly higher than those of the healthy human specimen. As a result of ROC analysis, in the colon cancer specimen, the area under curve was 0.838, and the specificity and the sensitivity when the cut-off value was 166.2 were 0.583 and 0.952, respectively. In the liver cancer specimens, the area under curve was 0.994, and the specificity and sensitivity when the cut-off value was 262.9 were 0.944 and 1.0, respectively. In the pancreatic cancer specimen, the area under curve was 0.944, and the specificity and sensitivity when the cut-off value was 262.2 were 0.944 and 0.756, respectively.

(C) Comparison with SDS Reduction Heat Treatment

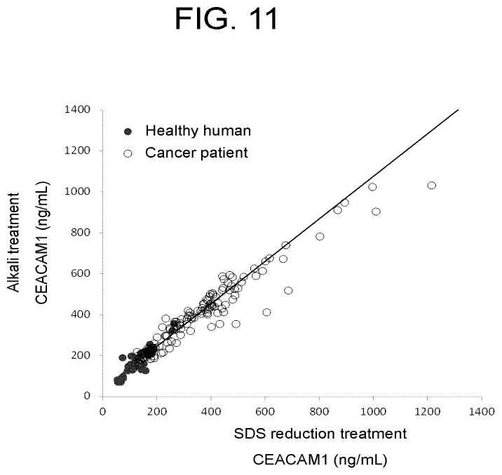

CEACAM1 concentrations in off-board SDS reduction heat treatment (X-axis) and on-board alkali treatment (Y-axis) were compared using 168 human-derived blood specimens (FIG. 11). The correlation coefficient of Pearson was highly positively correlated with r=0.967. The slope according to the regression of the Passing-Bablok method was 1.04.

Since SDS reduction heat treatment requires a specimen denaturation treatment at a high temperature (80° C.), a denaturation treatment step cannot be automated and the operation was complicated. By introducing an alkali treatment that does not require a high temperature treatment, it is possible to automate all steps including a denaturation treatment and to provide a simpler CEACAM1 measurement method.

### Example 11: Effect of Denaturation Treatment Using Autoantibody Model Specimen

In order to show the usefulness of the CEACAM1 measurement system including the denaturation treatment of the present invention, a measurement system not including the denaturation treatment was constructed using the antibody used in the ELISA kit from R&D Systems, and the reactivity to the autoantibody model specimen was compared.

### (A) Measurement of CEACAM1 Using Antibody of RD Company

10 μL of the specimen was mixed with 50 μL of a magnetic particle liquid (100 mM Tris-HCl, 20 mM EDTA3Na, 0.1% ProClin 300, 2% BSA, pH 7.5) containing magnetic particles bound to anti-CEACAM1 antibody 283324 (R&D Systems), and allowed to react for 8 minutes. The magnetic particles were magnetically collected and washed to remove components unbound to the magnetic particles, and 50 μL of a mixed liquid of 0.1 μg/mL biotin-labeled anti-CEACAM1 antibody BAF2244 (R&D Systems) and 0.2 μg/mL alkaline phosphatase (ALP)-labeled streptavidin diluted with a labeled form diluent (50 mM MES, 100 mM NaCl, 0.3 mM ZnCl₂, 1 mM MgCl₂, 2% BSA, 0.10% NaN₃, pH 6.8) was added thereto and reacted for 8 minutes. The magnetic particles were magnetically collected and washed to remove components unbound to the magnetic particles, and 200 μL of a substrate liquid (Lumipulse substrate liquid, manufactured by Fujirebio Inc.) containing AMPDD was added. The amount of luminescence due to the enzyme reaction was counted, and the CEACAM1 value in the specimen was calculated from the count using a calibration curve. For the calibration curve, CEACAM1 (R&D Systems) standard liquids corresponding to CEACAM1 amounts of 0, 8, 40, 200, and 1000 ng/mL were measured in the same manner as the specimen, and the calibration curve was prepared based on the amount of luminescence obtained for each standard liquid. All the processes of the present example were performed using an automated analyzing device LUMIPULSE L2400 (manufactured by Fujirebio Inc.).

### (B) Competitive Evaluation of CEACAM1 Polyclonal Antibody

A commercially available recombinant antigen CEACAM1 (R&D Systems) was diluted with PBS to 1 μg/mL. To a multimodule plate from Nunc Co., Ltd., 50 ML of the diluted antigen was added per well and allowed to stand at room temperature overnight. The antigen diluent was removed, and the plate was washed with PBS, then 100 μL of a blocking liquid (0.1% sodium caseinate, 150 mM NaCl, 1 mM EDTA, PBS) per well was added, and the plate was allowed to stand at room temperature for 1 hour.

The blocking liquid was removed, and 50 μL of each of various antibody solutions diluted to 0.1 μg/mL with a reaction liquid (0.1% sodium caseinate, 150 mM NaCl, 1 mM EDTA, PBS) or a solution obtained by adding 10 μg/mL of CEACAM1 polyclonal antibody (R&D Systems, AF2244) to various antibodies diluted to 0.1 μg/mL was added to each well, and the plate was allowed to stand at room temperature for 1 hour. The wells were washed with PBS containing 0.05% Tween20, 50 μL of an alkaline phosphatase (ALP)-labeled anti-mouse IgG Fc-specific antibody or ALP-labeled streptavidin was then added, and the wells were allowed to stand at room temperature for 1 hour. The wells were washed with PBS containing 0.05% Tween20, and then 50 UL of the AMPDD solution was added to each well to measure the amount of chemiluminescence.

Table 8 shows the binding rates of various antibodies by addition of the anti-CEACAM1 polyclonal antibody. BAF2244 and 283324, which are antibodies manufactured by R&D Systems, decreased the binding rate, and antibody A3054 of the present invention slightly increased the binding rate, but antibody A3073 and antibody A7004 decreased the binding rate. This result showed that both the measurement system using the antibody of R&D Systems and the measurement system using the antibody of the present invention were inhibited by the anti-CEACAM1 polyclonal antibody.

TABLE 8
Binding rates by addition of anti-CEACAM1 polyclonal antibody

| BAF2244 | 283324 | A3054 | A3073 | A7004 |
|---|---|---|---|---|
| 44.8% | 41.0% | 113.9% | 87.9% | 55.3% |

### (C) Comparison of Measurement Systems Using Autoantibody Model Specimen

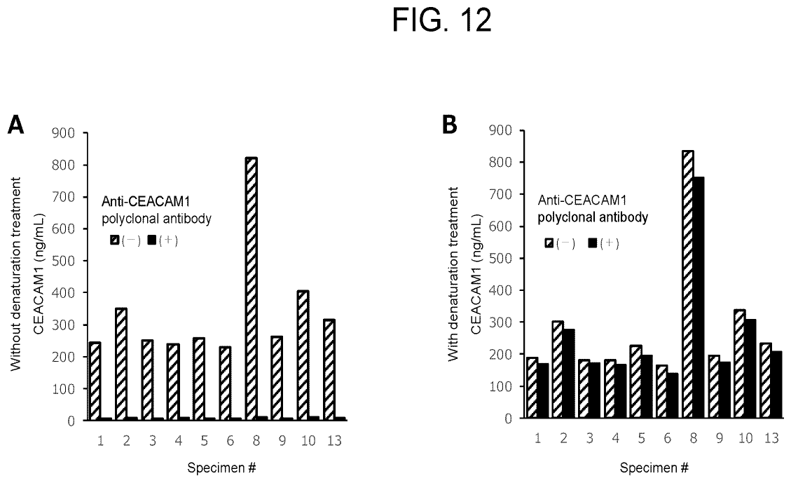

An anti-CEACAM1 polyclonal antibody (R&D Systems, AF2244) was added to a cancer specimen having a high value of CEACAM1 so as to be 30 μg/mL to prepare an autoantibody model specimen. CEACAM1 measurement “without denaturation treatment” or “with denaturation treatment” was performed on the specimens to which the anti-CEACAM1 polyclonal antibody was not added (−) and added (+). The “with denaturation treatment” was measured by CEACAM1 by the above-described alkali denaturation treatment, and the “without denaturation treatment” was measured by the above-described method for measuring CEACAM1 using an antibody from R&D Systems. The results are illustrated in FIG. 12. In the antibody from R&D Systems combination without denaturation treatment, the quantitative value of the addition (+) was reduced to about 3% as compared with the case where the polyclonal antibody was not added (−). On the other hand, the measurement system with denaturation treatment of the present invention was not greatly affected, and the quantitative value of about 90% was shown also in the added (+) specimen.

There are several reports on an autoantibody against CEA, and the frequency of their appearance in cancer patients is high (References 1 and 2). Since CEACAM1 belongs to CEA family, it is considered that there is a high possibility that an autoantibody will appear. In addition, since the CEA family containing CEACAM1 has high sequence and structural homology, the autoantibody against CEA may affect the CEACAM1 measurement system. From such a viewpoint, the CEACAM1 measurement system including the denaturation treatment of the present invention can avoid the influence of interfering substances such as an autoantibody, so that highly accurate diagnostic measurement can be performed.

### REFERENCES

1. K Albanopoulos et al., Am J Gastroenterol. 2000 April; 95 (4): 1056-61, PMID: 10763959, DOI: 10.1111/j.1572-0241.2000.01982.x
2. D Haidopoulos et al., Eur J Surg Oncol. 2000 December; 26(8): 742-6, PMID: 11087638, DOI: 10.1053/ejso.2000.0996

### Sequence Listing

[symbol]

1. A method for detecting CEACAM1, comprising:
(1) denaturing CEACAM1 in a CEACAM1-containing specimen to produce a denatured CEACAM1; and
(2) detecting CEACAM1 by a sandwich assay using both a capture antibody and a labeled antibody that are configured to recognize the denatured CEACAM1.

2. The method according to claim 1, wherein the denaturing is performed with a denaturant, heating, or a combination thereof.

3. The method according to claim 2, wherein the denaturant is at least one selected from the group consisting of an alkaline substance, a surfactant, and a reducing agent, and a combination thereof.

4. The method according to claim 1, wherein one of the capture antibody and the labeled antibody is an antibody configured to recognize an epitope represented by the amino acid sequence of PANSGRETIY (SEQ ID NO: 1) in CEACAM1.

5. The method according to claim 4, wherein a further one of the capture antibody and the labeled antibody is an antibody configured to recognize an epitope represented by the amino acid sequence of TESMP (SEQ ID NO: 2) in CEACAM1.

6. The method according to claim 4, wherein the other of the capture antibody and the labeled antibody is an antibody configured to recognize an epitope represented by the amino acid sequence of DTTYLWWINN (SEQ ID NO: 3) in CEACAM1.

7. The method according to claim 4, wherein the other of the capture antibody and the labeled antibody is an antibody configured to recognize an epitope represented by the amino acid sequence of EATGQFHVYP (SEQ ID NO: 4) in CEACAM1.

8. A method of detecting a cancer, comprising:
(1) measuring a CEACAM1 amount in a specimen collected from a subject by the method according to claim 1; and
(2) comparing the measured CEACAM1 amount to a reference value.

9. The method according to claim 8, wherein the cancer is at least one selected from the group consisting of colon cancer, liver cancer, pancreatic cancer, and a combination thereof.

10. A method for treating a cancer, comprising:
(1) measuring a CEACAM1 amount in a specimen collected from a subject by the method according to claim 1;
(2) comparing the measured CEACAM1 amount to a reference value;
(3) selecting a subject in which a CEACAM1 amount higher than a reference value has been measured; and
(4) administering an anti-cancer agent to the selected subject.

11. A kit for CEACAM1 detection, comprising:
(1) a denaturant;
(2) a capture antibody configured to recognize a denatured CEACAM1; and
(3) a labeled antibody configured to recognize a denatured CEACAM1.


SEQUENCE LISTING (53 sequences)

SEQ ID NO: 1 - protein, 10 aa, Homo sapiens
PANSGRETIY

SEQ ID NO: 2 - protein, 5 aa, Homo sapiens
TESMP

SEQ ID NO: 3 - protein, 10 aa, Homo sapiens
DTTYLWWINN

SEQ ID NO: 4 - protein, 10 aa, Homo sapiens
EATGQFHVYP

SEQ ID NO: 5 - protein, 137 aa, Homo sapiens
MKAIFVLKGSLDRDPELDHHHHHIEFGRTQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYP

SEQ ID NO: 6 - protein, 237 aa, Homo sapiens
MKAIFVLKGSLDRDPELDHHHHHIEFGRTQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPGSSGRGKFLNTAKDRNRWEEPDQQLYNVEATSYAGSGKFLNTAKDRNRWEEPDQQLYNVEATSYASGRGKFLNTAKDRNRWEEPDQQLYNVEATSYAGSG

SEQ ID NO: 7 - protein, 117 aa, Homo sapiens
MKAIFVLKGSLDRDPELDHHHHHIEFGRTPKPSISSNNSNPVEDKDAVAFTCEPETQDTTYLWWINNQSLPVSPRLQLSNGNRTLTLLSVTRNDTGPYECEIQNPVSANRSDPVTLN

SEQ ID NO: 8 - protein, 217 aa, Homo sapiens
MKAIFVLKGSLDRDPELDHHHHHIEFGRTPKPSISSNNSNPVEDKDAVAFTCEPETQDTTYLWWINNQSLPVSPRLQLSNGNRTLTLLSVTRNDTGPYECEIQNPVSANRSDPVTLNGSSGRGKFLNTAKDRNRWEEPDQQLYNVEATSYAGSGKFLNTAKDRNRWEEPDQQLYNVEATSYASGRGKFLNTAKDRNRWEEPDQQLYNVEATSYAGSG

SEQ ID NO: 9 - protein, 143 aa, Homo sapiens
QLTTESMPFNVAEGKEVLLLVHNLPQQLFGYSWYKGERVDGNRQIVGYAIGTQQATPGPANSGRETIYPNASLLIQNVTQNDTGFTYLWWINNQSLPVSPRLQLSNGNRTLTLLSVTRNDTGPYECEIQNPVSANRSDPVTLN

SEQ ID NO: 10 - protein, 177 aa, Homo sapiens
KLTIESMPLSVAEGKEVLLLVHNLPQHLFGYSWYKGERVDGNSLIVGYVIGTQQATPGAAYSGRETIYTNASLLIQNVTQNDIGFYTLQVIKSDLVNEEATGQFHVYQENAPGLPVGAVAGIVTGVLVGVALVAALVCFLLLAKTGRTSIQRDLKEQQPQALAPGRGPSHSSAFSMS

SEQ ID NO: 11 - protein, 175 aa, Homo sapiens
QFTIEALPSSAAEGKDVLLLACNISETIQAYYWHKGKTAEGSPLIAGYITDIQANIPGAAYSGRETVYPNGSLLFQNITLEDAGSYTLRTINASYDSDQATGQLHVHQNNVPGLPVGAVAGIVTGVLVGVALVAALVCFLLLSRTGRASIQRDLREQPPPASTPGHGPSHRSTFS

SEQ ID NO: 12 - protein, 198 aa, Homo sapiens
KLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILN

SEQ ID NO: 13 - protein, 198 aa, Homo sapiens
KLTIESTPFNVAEGKEVLLLAHNLPQNRIGYSWYKGERVDGNSLIVGYVIGTQQATPGPAYSGRETIYPNASLLIQNVTQNDTGFYTLQVIKSDLVNEEATGQFHVYPELPKPSISSNNSNPVEDKDAVAFTCEPEVQNTTYLWWVNGQSLPVSPRLQLSNGNMTLTLLSVKRNDAGSYECEIQNPASANRSDPVTLN

SEQ ID NO: 14 - protein, 199 aa, Homo sapiens
QTNIDVVPFNVAEGKEVLLVVHNESQNLYGYNWYKGERVHANYRIIGYVKNISQENAPGPAHNGRETIYPNGTLLIQNVTHNDAGFYTLHVIKENLVNEEVTRQFYVFSEPPKPSITSNNFNPVENKDIVVLTCQPETQNTTYLWWVNNQSLLVSPRLLLSTDNRTLVLLSATKNDIGPYECEIQNPVGASRSDPVTLN

SEQ ID NO: 15 - protein, 198 aa, Homo sapiens
QLTIEAVPSNAAEGKEVLLLVHNLPQDPRGYNWYKGETVDANRRIIGYVISNQQITPGPAYSNRETIYPNASLLMRNVTRNDTGSYTLQVIKLNLMSEEVTGQFSVHPETPKPSISSNNSNPVEDKDAVAFTCEPETQNTTYLWWVNGQSLPVSPRLQLSNGNRTLTLLSVTRNDVGPYECEIQNPASANFSDPVTLN

SEQ ID NO: 16 - DNA, 31 bp, synthetic construct
  source 1..31: mol_type = other DNA
gactcgtacgcagctcactactgaatccatg

SEQ ID NO: 17 - DNA, 34 bp, synthetic construct
  source 1..34: mol_type = other DNA
gactgtcgacttacgggtatacatggaactgtcc

SEQ ID NO: 18 - DNA, 31 bp, synthetic construct; identical to SEQ ID NO: 16
  source 1..31: mol_type = other DNA

SEQ ID NO: 19 - DNA, 33 bp, synthetic construct
  source 1..33: mol_type = other DNA
gactctcgagcccgggtatacatggaactgtcc

SEQ ID NO: 20 - DNA, 31 bp, synthetic construct
  source 1..31: mol_type = other DNA
gactcgtacgcccaagccctccatctccagc

SEQ ID NO: 21 - DNA, 34 bp, synthetic construct
  source 1..34: mol_type = other DNA
gactgtcgacttaattcaaggtgactgggtcact

SEQ ID NO: 22 - DNA, 31 bp, synthetic construct; identical to SEQ ID NO: 20
  source 1..31: mol_type = other DNA

SEQ ID NO: 23 - DNA, 33 bp, synthetic construct
  source 1..33: mol_type = other DNA
gactctcgagccattcaaggtgactgggtcact

SEQ ID NO: 24 - protein, 108 aa, synthetic construct
QLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYP

SEQ ID NO: 25 - protein, 98 aa, synthetic construct
QLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNE

SEQ ID NO: 26 - protein, 88 aa, synthetic construct
QLTTESMPFNVAEGKEVLLLVHNLPQQLFGYSWYKGERVDGNRQIVGYAIGTQQATPGPANSGRETIYPNASLLIQNVTQNDTGFYTL

SEQ ID NO: 27 - protein, 68 aa, synthetic construct
QLTTESMPFNVAEGKEVLLLVHNLPQQLFGYSWYKGERVDGNRQIVGYAIGTQQATPGPANSGRETIY

SEQ ID NO: 28 - protein, 58 aa, synthetic construct
QLTTESMPFNVAEGKEVLLLVHNLPQQLFGYSWYKGERVDGNRQIVGYAIGTQQATPG

SEQ ID NO: 29 - protein, 48 aa, synthetic construct
QLTTESMPFNVAEGKEVLLLVHNLPQQLFGYSWYKGERVDGNRQIVGY

SEQ ID NO: 30 - protein, 28 aa, synthetic construct
QLTTESMPFNVAEGKEVLLLVHNLPQQL

SEQ ID NO: 31 - protein, 18 aa, synthetic construct
QLTTESMPFNVAEGKEVL

SEQ ID NO: 32 - protein, 8 aa, synthetic construct
QLTTESMP

SEQ ID NO: 33 - protein, 15 aa, synthetic construct
TESMPFNVAEGKEVL

SEQ ID NO: 34 - protein, 12 aa, synthetic construct
MPFNVAEGKEVL

SEQ ID NO: 35 - protein, 9 aa, synthetic construct
NVAEGKEVL

SEQ ID NO: 36 - protein, 88 aa, synthetic construct
PKPSISSNNSNPVEDKDAVAFTCEPETQDTTYLWWINNQSLPVSPRLQLSNGNRTLTLLSVTRNDTGPYECEIQNPVSANRSDPVTLN

SEQ ID NO: 37 - protein, 68 aa, synthetic construct
PKPSISSNNSNPVEDKDAVAFTCEPETQDTTYLWWINNQSLPVSPRLQLSNGNRTLTLLSVTRNDTGP

SEQ ID NO: 38 - protein, 48 aa, synthetic construct
PKPSISSNNSNPVEDKDAVAFTCEPETQDTTYLWWINNQSLPVSPRLQ

SEQ ID NO: 39 - protein, 38 aa, synthetic construct
PKPSISSNNSNPVEDKDAVAFTCEPETQDTTYLWWINN

SEQ ID NO: 40 - protein, 28 aa, synthetic construct
PKPSISSNNSNPVEDKDAVAFTCEPETQ

SEQ ID NO: 41 - protein, 10 aa, synthetic construct
QLTTESMPFN

SEQ ID NO: 42 - protein, 10 aa, synthetic construct
KLTIESMPLS

SEQ ID NO: 43 - protein, 10 aa, synthetic construct
QFTIEALPSS

SEQ ID NO: 44 - protein, 10 aa, synthetic construct
KLTIESTPFN

SEQ ID NO: 45 - protein, 10 aa, synthetic construct
QTNIDVVPFN

SEQ ID NO: 46 - protein, 10 aa, synthetic construct
QLTIEAVPSN

SEQ ID NO: 47 - protein, 15 aa, synthetic construct
QATPGPANSGRETIY

SEQ ID NO: 48 - protein, 15 aa, synthetic construct
QATPGAAYSGRETIY

SEQ ID NO: 49 - protein, 15 aa, synthetic construct
ANIPGAAYSGRETVY

SEQ ID NO: 50 - protein, 15 aa, synthetic construct
QATPGPAYSGREIIY

SEQ ID NO: 51 - protein, 15 aa, synthetic construct
QATPGPAYSGRETIY

SEQ ID NO: 52 - protein, 15 aa, synthetic construct
ENAPGPAHNGRETIY

SEQ ID NO: 53 - protein, 15 aa, synthetic construct
QITPGPAYSNRETIY